(19) United States
(12) Patent Application Publication
(10) Pub. No.: US 2026/0090874 A1
(43) Pub. Date: Apr. 2, 2026

(54) DOUBLE-BRANCHING ASCENDING AORTIC STENT-GRAFT SYSTEMS

(71) Applicant: ENDOSPAN LTD., Herzilyia Pituach (IL)

(72) Inventors: Niv AVISAR, Ramat Gan (IL); Ian T. B. MCDOUGALL, Herzilya (IL)

(73) Assignee: ENDOSPAN LTD., Herzilyia Pituach (IL)

(21) Appl. No.: 19/110,140

(22) PCT Filed: Sep. 14, 2023
(86) PCT No.: PCT/IL2023/051002

§ 371 Date: Mar. 10, 2025

Related U.S. Application Data
(60) Provisional application No. 63/406,955, filed on Sep. 15, 2022.

(51) Int. Cl. A61F 2/07; A61F 2/06; A61F 2/954; A61F 2/966

(52) U.S. Cl. CPC A61F 2/07; A61F 2/954; A61F 2/966; A61F 2002/061; A61F 2002/065; A61F 2002/068

(57) ABSTRACT

A main stent-graft (22) is provided that includes an internal support channel (120) disposed within a generally tubular main fluid flow guide (28). When the internal support channel (120) is in a collapsed state, a distal channel-fluid-flow guide end opening (127B) of a channel fluid flow guide (128) faces radially inward. When the internal support channel (120) is in an expanded state and the main stent-graft (22) is in a radially-expanded deployment state, the distal channel-fluid-flow guide end opening (127B) faces at least partially distally within the main fluid flow guide (28). An elongate member (160A, 160B) is removably positioned passing sequentially through (a) a proximal main-fluid-flow guide end opening (27A), (b) a longitudinal portion (142) of a main fluid flow lumen (29) of the main-stent-graft fluid flow guide (28), (c) the distal channel-fluid-flow guide end opening (127B), and (d) a main-fluid-flow guide lateral opening (30) to outside the main fluid flow lumen (29). Other embodiments are also described.

### CROSS-REFERENCE TO RELATED APPLICATIONS

The present application claims priority from US Provisional Application 63/406,955, filed Sep. 15, 2022, which is assigned to the assignee of the present application and incorporated herein by reference.

### FIELD OF THE APPLICATION

The present application relates generally to prostheses and surgical methods, and specifically to tubular prostheses, including endovascular stent-grafts, and surgical techniques for using the prostheses to maintain patency of body passages such as blood vessels, and treating aneurysms, dissections, aortic ulcers, and intramural hematomas of arterial walls.

### BACKGROUND OF THE APPLICATION

Endovascular prostheses are sometimes used to treat acute aortic syndrome, which includes aortic aneurysm, aortic dissection, aortic ulcer (e.g., penetrating aortic ulcer (PAU), and intramural hematoma (IMH). Such treatment includes implanting a stent or stent-graft within the diseased vessel to bypass the anomaly. An aneurysm is a sac formed by the dilation of the wall of the artery. Aneurysms may be congenital, but are usually caused by disease or, occasionally, by trauma. Aortic aneurysms which commonly form between the renal arteries and the iliac arteries are referred to as abdominal aortic aneurysms (“AAAs”). Other aneurysms occur in the aorta, such as thoracic aortic aneurysms (“TAAs”), which may occur in one or more of the descending aorta, the ascending aorta, and the aortic arch.

Endo-Vascular Aneurysm Repair (EVAR) has transformed the practice of treatment of aortic aneurysms from an open surgical approach to a much less invasive surgical approach. The first step of an endovascular intervention usually requires introducing a delivery system into the vasculature of a patient.

WO 2014/108895 to Shalev et al. describes an endovascular system that includes an endovascular implant and a delivery tool. The implant is configured to assume a radially-compressed delivery state, and a radially-expanded deployment state. The delivery tool includes a proximal main delivery catheter, having a distal portion in which the implant is disposed while in the radially-compressed delivery state; and a distal restraining assembly, which includes a restraining-assembly tubular shaft disposed distal to the proximal main delivery catheter. The distal restraining assembly is configured to assume an engaged state, in which the distal restraining assembly prevents proximal displacement of the implant relative to the distal restraining assembly, and a disengaged state, in which the distal restraining assembly allows proximal displacement of the implant relative to the distal restraining assembly. Other embodiments are also described.

U.S. Pat. No. 8,267,988 to Hamer et al. describes an expandable prosthetic device and method of delivery that allows the initial placement of multiple guidewires into selected target sites. The prosthesis includes a main body device. This main body device has a separate side branch guidewire lumen that passes through the main body device and through a side opening in the main body device. As the main body device is advanced, the side opening is self-guided (by the side branch guidewire) and self-aligns to the side branch vessel ostium. The main body device is then deployed, leaving the side branch guidewire in place. A side branch device is then advanced along the side branch guidewire through the main body device, through the side wall opening and into the native side branch vessel. The side branch device can then be deployed to engage the main body device and the native side branch vessel.

U.S. Pat. No. 8,672,989 to Schreck et al. describes an endoluminal prosthesis system deployable in a region of a patient's vasculature having one or more branch vessels, having a main graft body having a first opening in a wall portion of the main graft body and a pre-loaded guidewire positioned inside the main graft body and advanced through the first opening. One or more branch grafts can be attached to the main graft body to cover one or more openings in the main graft body.

### SUMMARY OF THE APPLICATION

In some embodiments of the present application, a method is provided that comprises endovascularly introducing a vascular implant into vasculature of a patient through a vascular access site that is downstream of a first branch of an aortic arch. Thereafter, the vascular implant is advanced through the first branch to the aortic arch. Thereafter, the vascular implant is advanced from the aortic arch at least partially into a second branch of the aortic arch. Thereafter, the vascular implant is deployed at least partially in the second branch.

In some embodiments of the present application, a guidewire is deployed between (i) a first vascular access site that is downstream of a first branch of the aortic arch and (ii) a second vascular access site that is downstream of a second branch of the aortic arch, such that the guidewire is positioned partially within the first branch, partially within the aortic arch, and partially within the second branch. A vascular implant is advanced over the guidewire and deployed within the vasculature.

In some embodiments of the present application, a stent-graft system comprises a main stent-graft and an elongate member. The main stent-graft is configured to assume a radially-compressed delivery state and a radially-expanded deployment state. The main stent-graft comprises a flexible main-stent-graft stent member and a generally tubular main fluid flow guide, which is securely attached to and covers at least a portion of the main-stent-graft stent member. The main fluid flow guide is shaped so as to define proximal and distal main-fluid-flow guide end openings, a main fluid flow lumen therebetween, and a main-fluid-flow guide lateral opening. The main stent-graft further comprises an internal support channel, which is configured to assume expanded and collapsed states, and is disposed within the main fluid flow guide. The internal support channel comprises a generally tubular channel fluid flow guide, which is shaped so as to define proximal and distal channel-fluid-flow end openings. When the internal support channel is in the expanded state, the internal support channel defines a channel lumen between the proximal and the distal channel-fluid-flow end openings.

When the internal support channel is in the collapsed state, the distal channel-fluid-flow guide end opening faces radially inward. When the internal support channel is in the expanded state and the main stent-graft is in the radially-expanded deployment state, the distal channel-fluid-flow guide end opening faces at least partially distally within the main fluid flow guide.

The proximal channel-fluid-flow end opening is sealingly coupled to a perimeter of the main-fluid-flow guide lateral opening, such that the channel lumen is in fluid communication with outside the main fluid flow guide via the main-fluid-flow guide lateral opening when the internal support channel is in the expanded state. An external surface of the internal support channel is coupled to an internal surface of the main fluid flow guide such that the internal support channel runs alongside the internal surface of the main fluid flow guide distally from the main-fluid-flow guide lateral opening. Optionally, the internal support channel is also coupled to portions of the main-stent-graft stent member.

The elongate member is removably positioned passing sequentially through (a) the proximal main-fluid-flow guide end opening, (b) a longitudinal portion of the main fluid flow lumen, (c) the distal channel-fluid-flow guide end opening, and (d) the main-fluid-flow guide lateral opening to outside the main fluid flow lumen. The internal support channel is configured to automatically transition to the expanded state when not constrained in the collapsed state by the main stent-graft and not constrained in the collapsed state by the elongate member.

For some applications, the elongate member comprises a secondary guidewire tube that is shaped so as to define a secondary-guidewire-tube lumen for insertion therethrough of a secondary guidewire. For other applications, the elongate member comprises the secondary guidewire.

There is therefore provided, in accordance with an Inventive Concept 1 of the present invention, a stent-graft system comprising:
(i) a main stent-graft, which is configured to assume a radially-compressed delivery state and a radially-expanded deployment state, and which comprises:
   a flexible main-stent-graft stent member and a generally tubular main fluid flow guide, which (a) is securely attached to and covers at least a portion of the main-stent-graft stent member, and (b) is shaped so as to define proximal and distal main-fluid-flow guide end openings, a main fluid flow lumen therebetween, and a main-fluid-flow guide lateral opening; and
   an internal support channel, which (a) is configured to assume expanded and collapsed states, (b) is disposed within the main fluid flow guide, (c) comprises a generally tubular channel fluid flow guide, which is shaped so as to define (1) proximal and distal channel-fluid-flow end openings and (2) when the internal support channel is in the expanded state, a channel lumen between the proximal and the distal channel-fluid-flow end openings, wherein:
      (A) when the internal support channel is in the collapsed state, the distal channel-fluid-flow guide end opening faces radially inward, and
      (B) when the internal support channel is in the expanded state and the main stent-graft is in the radially-expanded deployment state, the distal channel-fluid-flow guide end opening faces at least partially distally within the main fluid flow guide,
   wherein the proximal channel-fluid-flow end opening is sealingly coupled to a perimeter of the main-fluid-flow guide lateral opening, such that the channel lumen is in fluid communication with outside the main fluid flow guide via the main-fluid-flow guide lateral opening when the internal support channel is in the expanded state; and
(ii) an elongate member, which is removably positioned passing sequentially through (a) the proximal main-fluid-flow guide end opening, (b) a longitudinal portion of the main fluid flow lumen, (c) the distal channel-fluid-flow guide end opening, and (d) the main-fluid-flow guide lateral opening to outside the main fluid flow lumen,
wherein the internal support channel is configured to automatically transition to the expanded state when not constrained in the collapsed state by the main stent-graft and not constrained in the collapsed state by the elongate member.

Inventive Concept 2. The stent-graft system according to Inventive Concept 1, for use with a secondary guidewire, wherein the elongate member comprises a secondary guidewire tube, which is shaped so as to define a secondary-guidewire-tube lumen for insertion therethrough of the secondary guidewire.

Inventive Concept 3. The stent-graft system according to Inventive Concept 2,
wherein a longitudinal portion of the secondary guidewire tube includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire tube is neither folded nor kinked.

Inventive Concept 4. The stent-graft system according to Inventive Concept 2,
wherein a longitudinal portion of the secondary guidewire tube includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion defines a directional path from the proximal main-fluid-flow guide end opening to the distal end of the second longitudinal sub-portion, and the directional path does not include any proximally-directed portions.

Inventive Concept 5. The stent-graft system according to Inventive Concept 2,
wherein a longitudinal portion of the secondary guidewire tube includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire tube has a smallest radius of curvature, at any location along the longitudinal portion, that is at least 0.15 cm.

Inventive Concept 6. The stent-graft system according to Inventive Concept 5, wherein the smallest radius of curvature is at least 1 cm.

Inventive Concept 7. The stent-graft system according to Inventive Concept 2, wherein the main stent-graft is configured such that the secondary guidewire tube is removable from the internal support channel while the main stent-graft is in the radially-compressed delivery state.

Inventive Concept 8. The stent-graft system according to Inventive Concept 7, wherein the main stent-graft is configured such that the secondary guidewire tube is removable from the internal support channel while the main stent-graft is in the radially-compressed delivery state and the secondary guidewire is inserted through the secondary-guidewire-tube lumen.

Inventive Concept 9. The stent-graft system according to Inventive Concept 1, wherein when the internal support channel is in the expanded state and the main stent-graft is in the radially-expanded deployment state, the distal channel-fluid-flow guide end opening is disposed distal to the main-fluid-flow guide lateral opening.

Inventive Concept 10. The stent-graft system according to Inventive Concept 1, wherein the internal support channel is configured such that when the internal support channel is in the collapsed state, a proximal-most point of the distal channel-fluid-flow guide end opening is disposed proximal of a distal-most point of the main-fluid-flow guide lateral opening.

Inventive Concept 11. The stent-graft system according to Inventive Concept 1, wherein the internal support channel is configured such that, during the automatic transition of the internal support channel from the collapsed state to the expanded state, a best-fit plane defined by the distal channel-fluid-flow guide end opening automatically swings:
from the distal channel-fluid-flow guide end opening facing at least partially radially inward,
to the distal channel-fluid-flow guide end opening facing at least partially distally.

Inventive Concept 12. The stent-graft system according to Inventive Concept 1,
wherein the main-stent-graft stent member comprises struts, and
wherein an external surface of the internal support channel is coupled to an internal surface of the main fluid flow guide by being coupled to one or more of the struts of the main-stent-graft stent member, such that the internal support channel runs alongside the internal surface of the main fluid flow guide distally from the main-fluid-flow guide lateral opening.

Inventive Concept 13. The stent-graft system according to Inventive Concept 1, wherein the elongate member comprises a secondary guidewire.

Inventive Concept 14. The stent-graft system according to Inventive Concept 13,
wherein a longitudinal portion of the secondary guidewire includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire is neither folded nor kinked.

Inventive Concept 15. The stent-graft system according to Inventive Concept 13,
wherein a longitudinal portion of the secondary guidewire includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion defines a directional path from the proximal main-fluid-flow guide end opening to the distal end of the second longitudinal sub-portion, and the directional path does not include any proximally-directed portions.

Inventive Concept 16. The stent-graft system according to Inventive Concept 13,
wherein a longitudinal portion of the secondary guidewire includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire has a smallest radius of curvature, at any location along the longitudinal portion, that is at least 0.15 cm.

Inventive Concept 17. The stent-graft system according to Inventive Concept 16, wherein the smallest radius of curvature is at least 1 cm.

Inventive Concept 18. The stent-graft system according to any one of Inventive Concepts 1-17, wherein the internal support channel further comprises a flexible channel stent member to which the channel fluid flow guide is securely attached.

Inventive Concept 19. The stent-graft system according to Inventive Concept 18, wherein the channel stent member comprises one or more stent struts, which are shaped so as to define a tongue-shaped stent strut that is securely attached to a portion of a perimeter of the distal channel-fluid-flow guide end opening.

Inventive Concept 20. The stent-graft system according to Inventive Concept 19, wherein the tongue-shaped stent strut is configured such that, during the automatic transition of the internal support channel from the collapsed state to the expanded state, a tip of the tongue-shaped stent strut automatically swings along a curved path from facing proximally to facing less proximally.

Inventive Concept 21. The stent-graft system according to Inventive Concept 19, wherein a minimal surface defined by the tongue-shaped stent strut faces:
at least partially distally when the internal support channel is in the expanded state, and
at least partially radially inward when the internal support channel is in the collapsed state.

Inventive Concept 22. The stent-graft system according to Inventive Concept 21, wherein the tongue-shaped stent strut is configured such that, during the automatic transition of the internal support channel to the expanded state, the tongue-shaped stent strut automatically swings:
from the minimal surface facing at least partially radially inward,
to the minimal surface facing at least partially distally.

Inventive Concept 23. The stent-graft system according to any one of Inventive Concepts 1-17,
wherein the stent-graft system further comprises a main delivery catheter having proximal and distal catheter ends,
wherein the main stent-graft is removably disposed within the main delivery catheter such that the main delivery catheter constrains the main stent-graft in the radially-compressed delivery state with the distal main-fluid-flow guide end opening facing the distal catheter end, thereby constraining the internal support channel in the collapsed state, and
wherein the elongate member is removably positioned passing sequentially through (a) the proximal main-fluid-flow guide end opening, (b) the longitudinal portion of the main fluid flow lumen, (c) the distal channel-fluid-flow guide end opening, (d) the main-fluid-flow guide lateral opening to outside the main fluid flow lumen within the main delivery catheter, and (e) the distal catheter end.

Inventive Concept 24. The stent-graft system according to Inventive Concept 23, wherein a longitudinal portion of the elongate member extends between the proximal main-fluid-flow guide end opening of the radially-compressed main stent-graft and the distal catheter end, and has a smallest radius of curvature, at any location along the longitudinal portion, that is at least 0.15 cm.

Inventive Concept 25. The stent-graft system according to Inventive Concept 24, wherein the smallest radius of curvature is at least 1 cm.

Inventive Concept 26. The stent-graft system according to Inventive Concept 23, wherein a longitudinal portion of the elongate member extends between the proximal main-fluid-flow guide end opening of the radially-compressed main stent-graft and the distal catheter end, and is neither folded nor kinked.

Inventive Concept 27. The stent-graft system according to Inventive Concept 23, wherein a longitudinal portion of the elongate member defines a directional path from the proximal main-fluid-flow guide end opening of the radially-compressed main stent-graft to the distal catheter end, and the directional path does not include any proximally-directed portions.

There is further provided, in accordance with an Inventive Concept 28 of the present invention, a method comprising:
providing a main stent-graft, which is configured to assume a radially-compressed delivery state and a radially-expanded deployment state, and which includes:
   a flexible main-stent-graft stent member and a generally tubular main fluid flow guide, which (a) is securely attached to and covers at least a portion of the main-stent-graft stent member, and (b) is shaped so as to define proximal and distal main-fluid-flow guide end openings, a main fluid flow lumen therebetween, and a main-fluid-flow guide lateral opening; and
   an internal support channel, which (a) is configured to assume expanded and collapsed states, (b) is disposed within the main fluid flow guide, (c) includes a generally tubular channel fluid flow guide, which is shaped so as to define (1) proximal and distal channel-fluid-flow guide end openings and (2) when the internal support channel is in the expanded state, a channel lumen between the proximal and the distal channel-fluid-flow guide end openings,
   wherein the proximal channel-fluid-flow guide end opening is sealingly coupled to a perimeter of the main-fluid-flow guide lateral opening, such that the channel lumen is in fluid communication with outside the main fluid flow guide via the main-fluid-flow guide lateral opening when the internal support channel is in the expanded state;
providing an elongate member that is removably positioned passing sequentially through (a) the proximal main-fluid-flow guide end opening, (b) a longitudinal portion of main fluid flow lumen, (c) the distal channel-fluid-flow guide end opening, and (d) the main-fluid-flow guide lateral opening to outside the main fluid flow lumen;
introducing the main stent-graft into vasculature of a patient while (a) the main stent-graft is in the radially-compressed delivery state, and (b) the internal support channel is in the collapsed state, in which the distal channel-fluid-flow guide end opening faces radially inward; and
thereafter, transitioning the main stent-graft to the radially-expanded deployment state,
   wherein the internal support channel is configured to automatically transition to the expanded state when not constrained in the collapsed state by the main stent-graft and not constrained in the collapsed state by the elongate member, and wherein when the internal support channel is in the expanded state, the distal channel-fluid-flow guide end opening faces at least partially distally within the main fluid flow guide.

Inventive Concept 29. The method according to Inventive Concept 28,
wherein the elongate member is a secondary guidewire tube, and
wherein the method further comprises, before introducing the main stent-graft into the vasculature, inserting a secondary guidewire through a secondary-guidewire-tube lumen of the secondary guidewire tube.

Inventive Concept 30. The method according to Inventive Concept 29, further comprising, after the internal support channel automatically transitions to the expanded state in which the distal channel-fluid-flow guide end opening faces at least partially distally within the main fluid flow guide:
advancing a branching stent-graft over the secondary guidewire (a) from a vascular access site to within the main stent-graft via the distal main-fluid-flow guide end opening, (b) into the distal channel-fluid-flow guide end opening, (c) through the channel fluid flow guide, and (d) out of the main-fluid-flow guide lateral opening via the proximal channel-fluid-flow end opening.

Inventive Concept 31. The method according to Inventive Concept 29, further comprising, after inserting the secondary guidewire through the secondary-guidewire-tube lumen and before introducing the main stent-graft into the vasculature:
removing the secondary guidewire tube from the internal support channel while the main stent-graft is in the radially-compressed delivery state outside a body of the patient, such that the secondary guidewire remains positioned passing sequentially through (a) the proximal main-fluid-flow guide end opening, (b) the longitudinal portion of the main fluid flow lumen, (c) the distal channel-fluid-flow guide end opening, and (d) the main-fluid-flow guide lateral opening to outside the main fluid flow lumen.

Inventive Concept 32. The method according to Inventive Concept 29, wherein inserting the secondary guidewire through the secondary-guidewire-tube lumen comprises inserting the secondary guidewire through the secondary-guidewire-tube lumen while the main stent-graft is in the radially-compressed delivery state.

Inventive Concept 33. The method according to Inventive Concept 32, wherein inserting the secondary guidewire through the secondary-guidewire-tube lumen comprises inserting the secondary guidewire through the secondary-guidewire-tube lumen while the main stent-graft is outside a body of the patient.

Inventive Concept 34. The method according to Inventive Concept 28, wherein when the internal support channel is in the expanded state and the main stent-graft is in the radially-expanded deployment state, the distal channel-fluid-flow guide end opening is disposed distal to the main-fluid-flow guide lateral opening.

Inventive Concept 35. The method according to Inventive Concept 28,
wherein the elongate member is a secondary guidewire, and
wherein the method further comprises, before introducing the main stent-graft into the vasculature, deploying the secondary guidewire between two vascular access sites.

Inventive Concept 36. The method according to Inventive Concept 35, further comprising, after the internal support channel automatically transitions to the expanded state in which the distal channel-fluid-flow guide end opening faces at least partially distally within the main fluid flow guide:
advancing a branching stent-graft over the secondary guidewire (a) from a vascular access site to within the main stent-graft via the distal main-fluid-flow guide end opening, (b) into the distal channel-fluid-flow guide end opening, (c) through the channel fluid flow guide, and (d) out of the main-fluid-flow guide lateral opening via the proximal channel-fluid-flow end opening.

There is still further provided, in accordance with an Inventive Concept 37 of the present invention, a method comprising:
endovascularly introducing a vascular implant into vasculature of a patient through a vascular access site that is downstream of a first branch of an aortic arch;
thereafter, advancing the vascular implant through the first branch to the aortic arch;
thereafter, advancing the vascular implant from the aortic arch at least partially into a second branch of the aortic arch; and
thereafter, deploying the vascular implant at least partially in the second branch.

Inventive Concept 38. The method according to Inventive Concept 37, wherein the first branch is a brachiocephalic artery, and wherein the second branch is selected from the group of branches consisting of: a left common carotid artery (LCCA) and a left subclavian artery (LSA).

Inventive Concept 39. The method according to Inventive Concept 37,
wherein the vascular access site is a first vascular access site,
wherein the method further comprises deploying a guidewire between the first vascular access site and a second vascular access site that is downstream of the second branch, such that the guidewire is positioned partially within the first branch, partially
within the aortic arch, and partially within the second branch, and wherein advancing the vascular implant through the first branch to the aortic arch and from the aortic arch at least partially into the second branch comprises:
   advancing the vascular implant over the guidewire through the first branch to the aortic arch; and
   thereafter, advancing the vascular implant over the guidewire from the aortic arch at least partially into the second branch.

Inventive Concept 40. The method according to Inventive Concept 37,
wherein endovascularly introducing the vascular implant into the vasculature comprises endovascularly introducing the vascular implant into the vasculature while the vascular implant is disposed in a radially-compressed delivery state within a portion of a delivery catheter,
wherein advancing the vascular implant through the first branch to the aortic arch and from the aortic arch at least partially into the second branch comprises advancing the portion of the delivery catheter through the first branch to the aortic arch and from the aortic arch at least partially into the second branch, and
wherein deploying the vascular implant at least partially in the second branch comprises releasing the vascular implant from the portion of the delivery catheter, such that the vascular implant transitions to a radially-expanded deployment state at least partially in the second branch.

Inventive Concept 41. The method according to Inventive Concept 37, wherein the vascular implant includes a stent-graft, which includes a flexible stent member and a generally tubular fluid flow guide, which is securely attached to and covers at least a portion of the stent member.

Inventive Concept 42. The method according to Inventive Concept 41,
wherein the stent-graft is a branching stent-graft, wherein the stent member is a branching-stent-graft stent member, and wherein the fluid flow guide is a branching-stent-graft fluid flow guide,
wherein the method further comprises, before advancing the vascular implant through the first branch to the aortic arch, deploying a main stent-graft at least partially in the aortic arch, such that a main-fluid-flow guide lateral opening defined by a generally tubular main-stent-graft fluid flow guide of the main stent-graft is oriented toward the second branch, the main stent-graft further including a flexible main-stent-graft stent member to which the main-stent-graft fluid flow guide is securely attached so as to cover at least a portion of the main-stent-graft stent member,
wherein advancing the vascular implant through the first branch to the aortic arch comprises advancing the branching stent-graft through the first branch to within the main stent-graft,
wherein advancing the vascular implant from the aortic arch at least partially into the second branch comprises advancing the branching stent-graft from within the main stent-graft partially into the second branch via the main-fluid-flow guide lateral opening, and
wherein deploying the vascular implant at least partially in the second branch comprises deploying the branching stent-graft partially in the second branch external to the main-fluid-flow guide lateral opening and partially inside the main stent-graft.

Inventive Concept 43. The method according to Inventive Concept 41, wherein the aortic arch suffers from acute aortic syndrome.

Inventive Concept 44. The method according to Inventive Concept 43, wherein a wall of the aortic arch suffers from an aneurysm.

Inventive Concept 45. The method according to Inventive Concept 43, wherein a wall of the aortic arch suffers from a dissection.

Inventive Concept 46. The method according to Inventive Concept 43, wherein a wall of the aortic arch suffers from a penetrating aortic ulcer (PAU).

Inventive Concept 47. The method according to Inventive Concept 43, wherein a wall of the aortic arch suffers from an intramural hematoma (IMH).

There is additionally provided, in accordance with an Inventive Concept 48 of the present invention, a method comprising:
deploying a guidewire between (i) a first vascular access site that is downstream of a first branch of an aortic arch of a patient and (ii) a second vascular access site that is downstream of a second branch of the aortic arch, such that the guidewire is positioned partially within the first branch, partially within the aortic arch, and partially within the second branch;
advancing a vascular implant over the guidewire; and
deploying the vascular implant within vasculature of the patient.

Inventive Concept 49. The method according to Inventive Concept 48, wherein the first branch is a brachiocephalic artery, and wherein the second branch is selected from the group of branches consisting of: a left common carotid artery (LCCA) and a left subclavian artery (LSA).

Inventive Concept 50. The method according to Inventive Concept 49, wherein the first vascular access site is on a right axillary artery (RAA).

Inventive Concept 51. The method according to Inventive Concept 49, wherein the second branch is the LCCA, and the second vascular access site is on the LCCA.

Inventive Concept 52. The method according to Inventive Concept 48, wherein deploying the guidewire between the first vascular access site the second vascular access site, such that the guidewire is positioned partially within the first branch, partially within the aortic arch, and partially within the second branch, comprises:
deploying the guidewire between (i) the second vascular access site and (ii) a third vascular access site that is downstream of a descending aorta, such that:
   (a) the guidewire is positioned partially within the second branch, partially within the aortic arch, and partially within the descending aorta,
   (b) a first end portion of the guidewire passes out of the vasculature at the second vascular access site, and
   (c) a second end portion of the guidewire, opposite the first end portion, passes out of the vasculature at the third vascular access site; and
forming the second end portion of the guidewire into a loop outside a body of the patient by reinserting the second end portion of the guidewire into the vasculature through the third vascular access site; and
thereafter, advancing the second end portion of the guidewire through the descending aorta, through a portion of the aortic arch, through at least a portion of the first branch, and out of the first vascular access site, such that the loop is drawn into the vasculature via the third vascular access site.

Inventive Concept 53. The method according to Inventive Concept 52,
wherein the guidewire is a secondary guidewire, and wherein deploying the guidewire between the second vascular access site and the third vascular access site comprises deploying the secondary guidewire between the second vascular access site and the third vascular access site, and
wherein deploying the guidewire between the first vascular access site the second vascular access site comprises deploying the secondary guidewire between the first vascular access site the second vascular access site by:
   deploying a primary guidewire between the first vascular access site and the third vascular access site, such that (a) a first end portion of the primary guidewire passes out of the vasculature at the first vascular access site, and (b) a second end portion of the primary guidewire, opposite the first end portion of the primary guidewire, passes out of the vasculature at the third vascular access site,
   deploying a through-and-through catheter over the primary guidewire between the first vascular access site and the third vascular access site, such that (a) a first end portion of the through-and-through catheter passes out of the vasculature at the first vascular access site, and (b) a second end portion of the through-and-through catheter, opposite the first end portion of the through-and-through catheter, passes out of the vasculature at the third vascular access site,
   thereafter, removing the primary guidewire from the through-and-through catheter, and
   thereafter, advancing the second end portion of the secondary guidewire through the through-and-through catheter and out of the first vascular access site, such that the loop is drawn into the vasculature via the third vascular access site.

Inventive Concept 54. The method according to Inventive Concept 53, wherein the method further comprises, after deploying the primary guidewire between the first vascular access site and the third vascular access site and before deploying the through-and-through catheter over the primary guidewire and the vascular implant within the vasculature:
deploying, over the primary guidewire, a main stent-graft at least partially in the aortic arch, such that a main-fluid-flow guide lateral opening defined by a main-stent-graft fluid flow guide of the main stent-graft is oriented toward the second branch, the main stent-graft further including a flexible main-stent-graft stent member to which the main-stent-graft fluid flow guide is securely attached so as to cover at least a portion of main-stent-graft stent member.

Inventive Concept 55. The method according to Inventive Concept 54, wherein deploying main stent-graft comprises:
introducing a delivery catheter into the vasculature through the third vascular access site and advancing the delivery catheter over the primary guidewire and the secondary guidewire while the main stent-graft is disposed in a radially-compressed delivery state within a portion of the delivery catheter; and
releasing the main stent-graft from the portion of the delivery catheter, such that the main stent-graft transitions to a radially-expanded deployment state at least partially in the aortic arch.

Inventive Concept 56. The method according to Inventive Concept 54,
wherein the vascular implant includes a branching stent-graft, which includes a flexible branching-stent-graft stent member and a generally tubular branching-stent-graft fluid flow guide, which is securely attached to and covers at least a portion of the branching-stent-graft stent member,
wherein deploying the vascular implant within vasculature of the patient comprises, after deploying the main stent-graft at least partially in the aortic arch, advancing the branching stent-graft over the secondary guidewire from the first vascular access site to within the main stent-graft, from within the main stent-graft, and partially into the second branch via the main-fluid-flow guide lateral opening, such that the branching stent-graft is partially in the second branch external to the main-fluid-flow guide lateral opening and partially inside the main stent-graft.

Inventive Concept 57. The method according to Inventive Concept 48,
wherein advancing the vascular implant comprises advancing the vascular implant over the guidewire while the vascular implant is disposed in a radially-compressed delivery state within a portion of a delivery catheter,
wherein deploying the vascular implant within the vasculature comprises releasing the vascular implant from the portion of the delivery catheter, such that the vascular implant transitions to a radially-expanded deployment state within the vasculature.

Inventive Concept 58. The method according to Inventive Concept 48, wherein the vascular implant includes a stent-graft, which includes a flexible stent member and a generally tubular fluid flow guide, which is securely attached to and covers at least a portion of the stent member.

Inventive Concept 59. The method according to Inventive Concept 58, wherein the aortic arch suffers from acute aortic syndrome.

Inventive Concept 60. The method according to Inventive Concept 59, wherein a wall of the aortic arch suffers from an aneurysm.

Inventive Concept 61. The method according to Inventive Concept 59, wherein a wall of the aortic arch suffers from a dissection.

Inventive Concept 62. The method according to Inventive Concept 59, wherein a wall of the aortic arch suffers from a penetrating aortic ulcer (PAU).

Inventive Concept 63. The method according to Inventive Concept 59, wherein a wall of the aortic arch suffers from an intramural hematoma (IMH).

Inventive Concept 64. The method according to Inventive Concept 48, wherein advancing the vascular implant over the guidewire comprises advancing the vascular implant over the guidewire from the first vascular access site to the aortic arch, and from the aortic arch at least partially into the second branch.

Inventive Concept 65. The method according to Inventive Concept 64,
wherein the vascular implant includes a branching stent-graft, which includes a flexible branching-stent-graft stent member and a tubular fluid branching-stent-graft generally flow guide, which is securely attached to and covers at least a portion of the branching-stent-graft stent member,
wherein the method further comprises, before deploying the vascular implant within the vasculature, deploying a main stent-graft at least partially in the aortic arch, such that a main-fluid-flow guide lateral opening defined by a tubular fluid main-stent-graft fluid flow guide of the main stent-graft is oriented toward the second branch, the main stent-graft further including a flexible main-stent-graft stent member to which the main-stent-graft fluid flow guide is securely attached so as to cover at least a portion of the main-stent-graft stent member,
wherein advancing the vascular implant over the guidewire from the first vascular access site to the aortic arch comprises advancing the branching stent-graft from the first vascular access site to within the main stent-graft,
wherein advancing the vascular implant from the aortic arch at least partially into the second branch comprises advancing the branching stent-graft from within the main stent-graft, over the guidewire, partially into the second branch via the main-fluid-flow guide lateral opening, and
wherein deploying the vascular implant within the vasculature comprises deploying the branching stent-graft partially in the second branch external to the main-fluid-flow guide lateral opening and partially inside the main stent-graft.

The present invention will be more fully understood from the following detailed description of embodiments thereof, taken together with the drawings, in which:

### BRIEF DESCRIPTION OF THE DRAWINGS

FIGS. 1A-B are schematic illustrations of a vascular implant assembly in disassembled and assembled states, respectively, in accordance with an application of the present invention;

FIGS. 1C-D are schematic illustrations of another configuration of the vascular implant assembly of FIGS. 1A-B in disassembled and assembled states, respectively, in accordance with an application of the present invention;

FIGS. 2A-O are schematic illustrations of several stages of a method for deploying one or more vascular implants, in accordance with an application of the present invention;

FIG. 3 is a schematic illustration of an alternative implantation of the vascular implant assembly of FIGS. 1A-B in vasculature, in accordance with an application of the present invention;

FIGS. 4A-C are side-view schematic illustrations of an internal support channel of a main stent-graft of the vascular implant assembly of FIGS. 1A-B in an expanded state, a partially-collapsed state, and a collapsed state, respectively, in accordance with an application of the present invention;

FIGS. 4D-F are side-view schematic illustrations of the internal support channel of the main stent-graft of the vascular implant assembly of FIGS. 1A-B in the expanded state, the partially-collapsed state, and the collapsed state, respectively, in accordance with another application of the present invention;

FIG. 5 is a schematic illustration of a flexible channel stent member of the internal support channel of FIGS. 4A-C in an expanded state, from a partially distal and partially side view, in accordance with an application of the present invention;

FIGS. 6A-C are schematic illustrations of several stages of a portion of a method for deploying a main stent-graft and a branching stent-graft, in accordance with an application of the present invention; and

FIG. 7 is a schematic illustration of a configuration of a main stent-graft of the vascular implant assembly of FIGS. 1A-B disposed in a radially-compressed delivery state within a portion of a main delivery catheter, in accordance with an application of the present invention.

### DETAILED DESCRIPTION OF APPLICATIONS

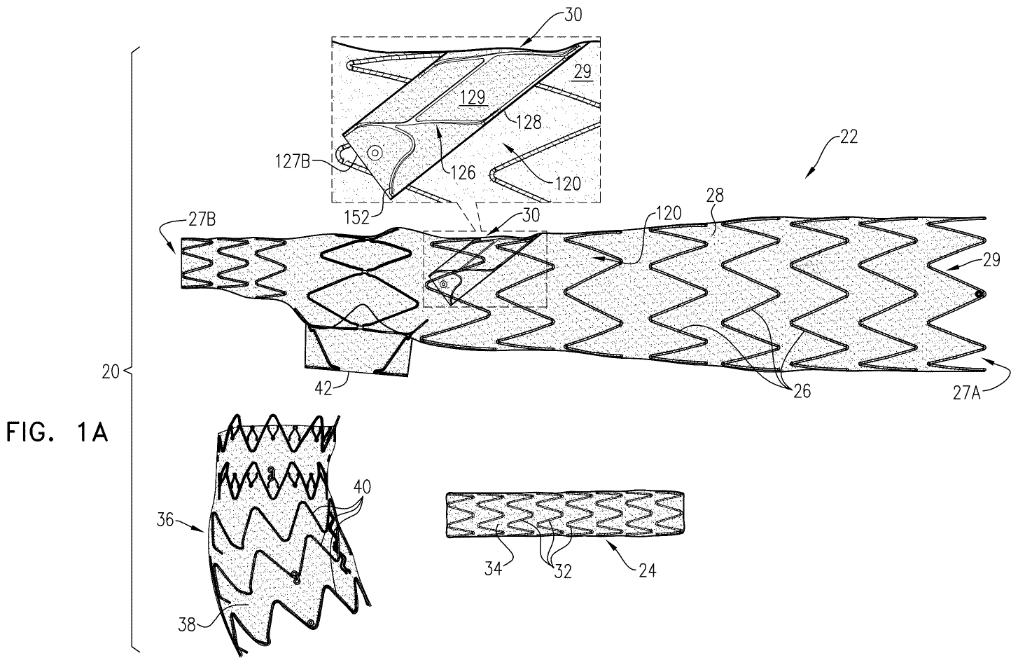

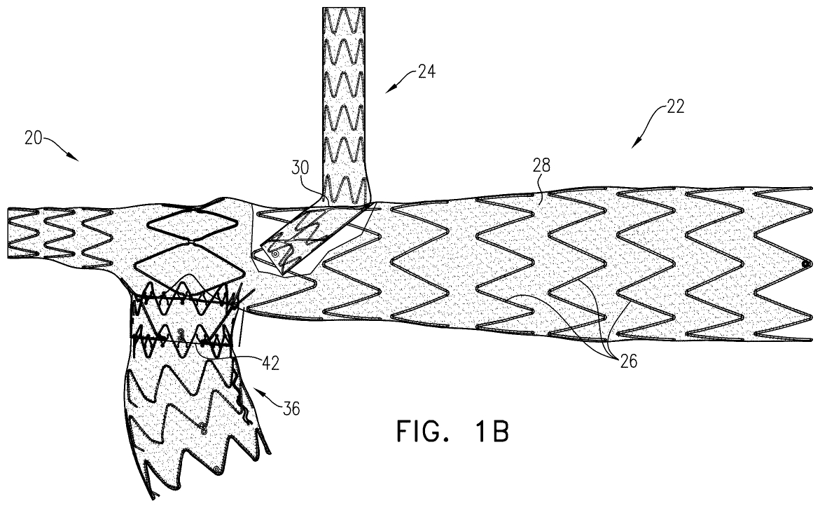

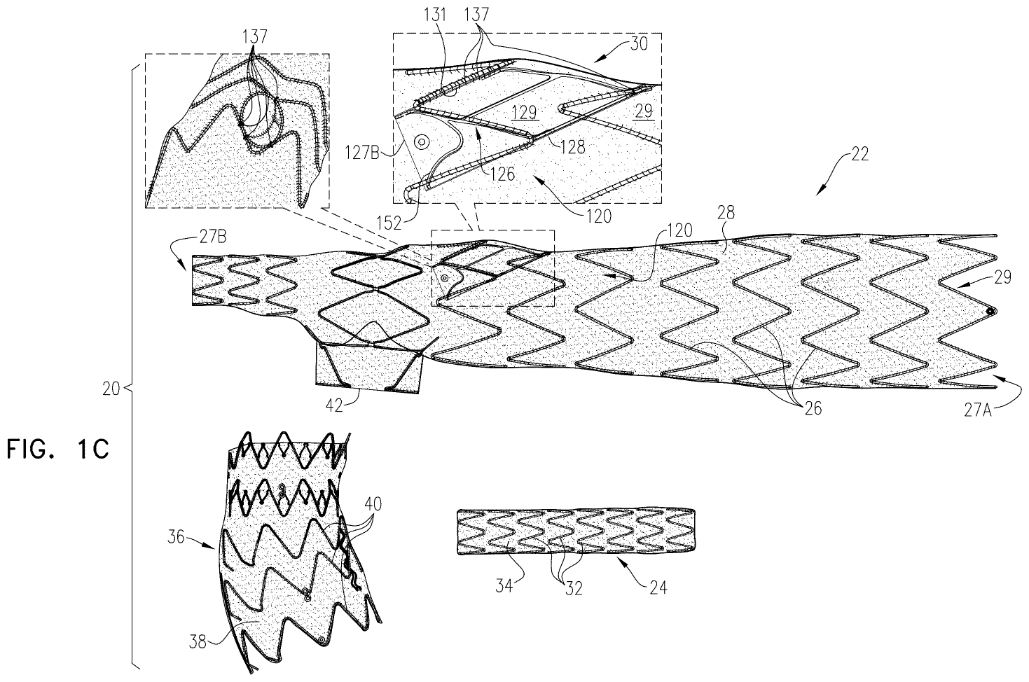

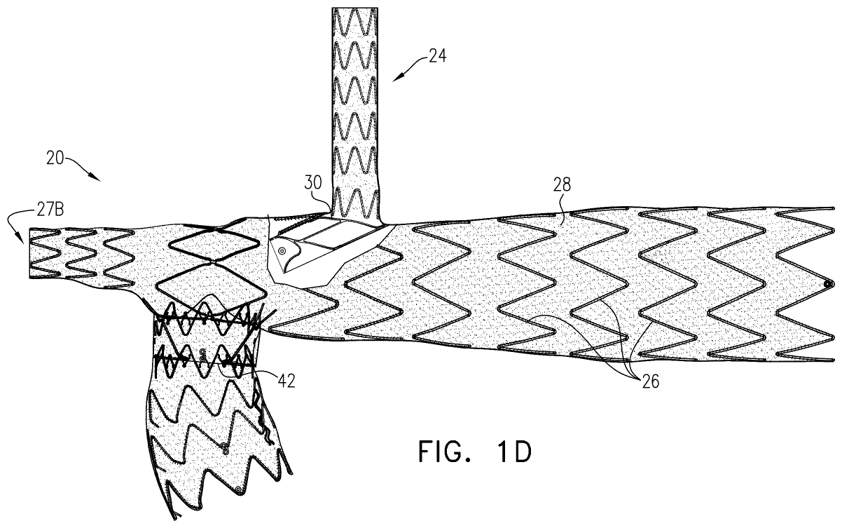

FIGS. 1A-B are schematic illustrations of a vascular implant assembly 20 in disassembled and assembled states, respectively, in accordance with an application of the present invention. Vascular implant assembly 20 typically comprises a main stent-graft 22 and a branching stent-graft 24. Both main stent-graft 22 and branching stent-graft 24 are shown in radially-expanded deployment states in FIGS. 1A-B.

FIGS. 1C-D are schematic illustrations of another configuration of vascular implant assembly in disassembled and assembled states, respectively, in accordance with an application of the present invention. Both main stent-graft 22 and branching stent-graft 24 are shown in radially-expanded deployment states in FIGS. 1C-D. The difference between the configurations of FIGS. 1A-B and 1C-D is described hereinbelow with reference to FIGS. 1C-D.

Main stent-graft 22 typically comprises a flexible main-stent-graft stent member 26 and a generally tubular fluid flow guide 28. Generally tubular fluid flow guide 28 is securely attached to main-stent-graft stent member 26, such as by suturing or stitching, so as to cover at least a portion of main-stent-graft stent member 26. The stent member may be attached to an internal surface and/or an external surface of the fluid flow guide. Optionally, a portion of the stent struts of the stent member may be attached (e.g., sutured) to the internal surface, and another portion to the external surface. Main-stent-graft fluid flow guide 28 is shaped so as to define proximal and distal main-fluid-flow guide end openings 27A and 27B, a main fluid flow lumen 29 therebetween, and a main-fluid-flow guide lateral opening 30.

For some applications, main-stent-graft fluid flow guide 28 has one or more of the following dimensions when unconstrained (by a deployment catheter, by anatomy, or otherwise) in the radially-expanded deployment state:
a length of at least 15 cm, no more than 30 cm, and/or 15-30 cm,
a greatest diameter of at least 1 cm, no more than 5 cm, and/or 1-5 cm,
an area of lateral opening 30 of at least 0.5 cm2, no more than 2.1 cm2, and/or 0.5-2.1 cm2,
a perimeter of lateral opening 30 of at least 2 cm, no more than 6.5 cm, and/or 2-6.5 cm,
a perimeter of proximal main-fluid-flow guide end opening 27A of at least 10 cm, no more than 15 cm, and/or 10-15 cm, and/or
a perimeter of distal main-fluid-flow guide end opening 27B of at least 1.6 cm, no more than 4.5 cm, and/or 1.6-4.5 cm.

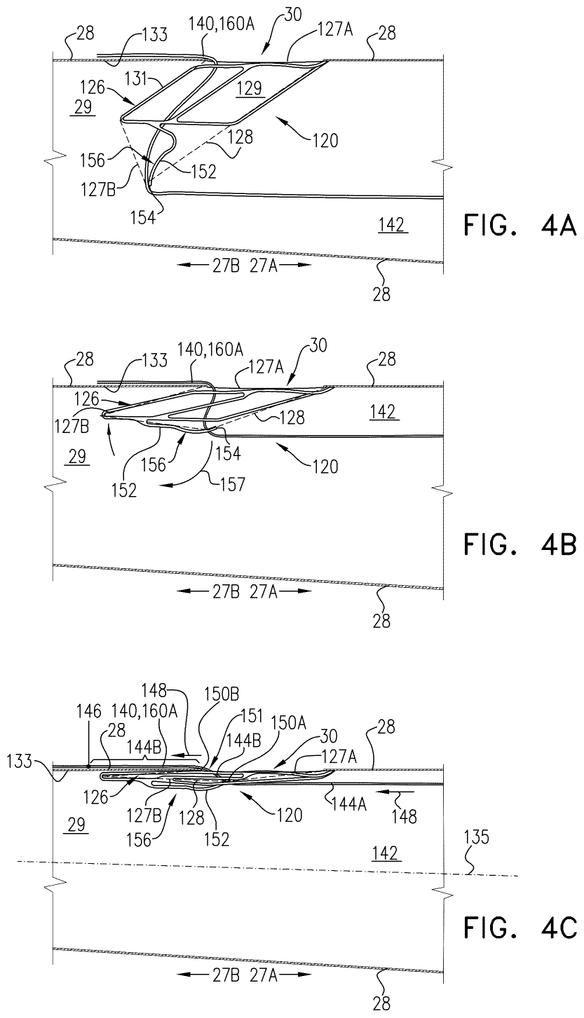

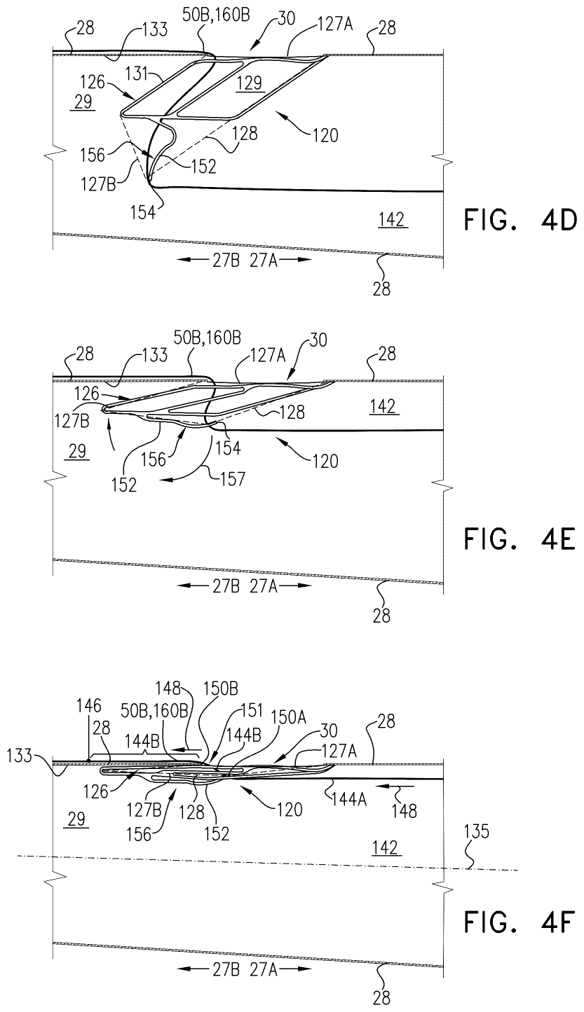

Branching stent-graft 24 typically comprises a flexible branching-stent-graft stent member 32 and a generally tubular branching-stent-graft fluid flow guide 34. Branching-stent-graft fluid flow guide 34 is securely attached to branching-stent-graft stent member 32, such as by suturing or stitching, so as to cover at least a portion of branching-stent-graft stent member 32. The stent member may be attached to an internal surface and/or an external surface of the fluid flow guide. Optionally, a portion of the stent struts of the stent member may be attached (e.g., sutured) to the internal surface, and another portion to the external surface. Further optionally, branching-stent-graft fluid flow guide 34 comprises two layers of graft bonded together, between which branching-stent-graft stent member 32 is sandwiched, thereby encapsulating branching-stent-graft stent member 32 in graft material of branching-stent-graft fluid flow guide 34. Branching stent-graft 24 is configured to be disposed through main-fluid-flow guide lateral opening 30 so as to form a blood-tight seal between branching-stent-graft fluid flow guide 34 and:
the portion of main-stent-graft fluid flow guide 28 that defines the perimeter of main-fluid-flow guide lateral opening 30, such as shown in FIG. 1B, and/or
an internal surface of tubular channel fluid flow guide 128 of internal support channel 120, in applications in which main stent-graft 22 comprises internal support channel 120, such as shown in FIG. 1B, and such as described in detail hereinbelow with reference to FIGS. 4A-C, 4D-F, 5, 6A-C, and 7.

For some applications, main stent-graft 22 is configured to be positioned partially in a descending aorta 66, partially in an aortic arch 62, and partially in a first branch 60 of aortic arch 62. For example, first branch 60 may be a brachiocephalic artery (BCT) 64. As used in the present application, including the claims and Inventive Concepts, a “branch” of the aortic arch means an artery that branches from the aortic arch, i.e., BCT 64, a left common carotid artery (LCCA) 72, or a left subclavian artery (LSA) 74. As used in the present application, including the claims and Inventive Concepts, a “branch” of the aortic arch does not mean the ostium of the branch (i.e., the opening of the branch off of the aortic arch). Thus, “downstream” of a “branch” of the aortic arch means downstream along the branching artery, rather than downstream along the continuation of the aorta.

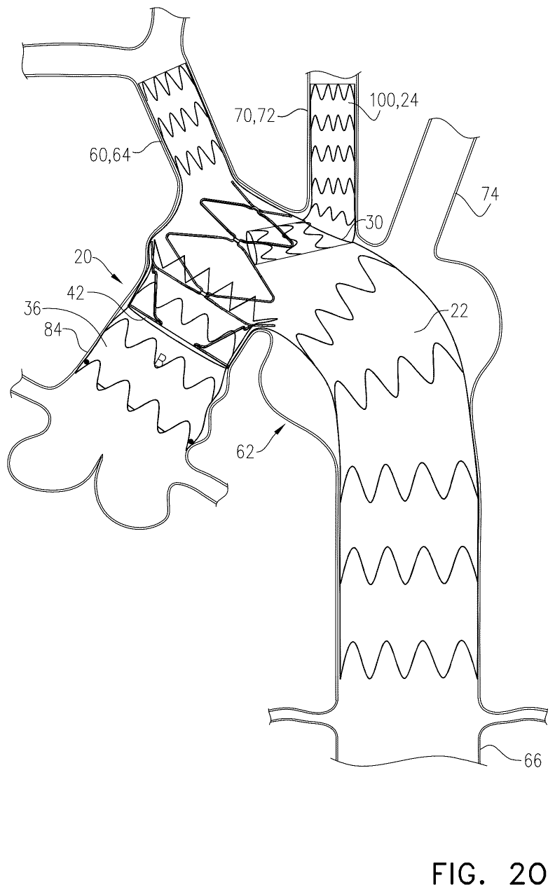

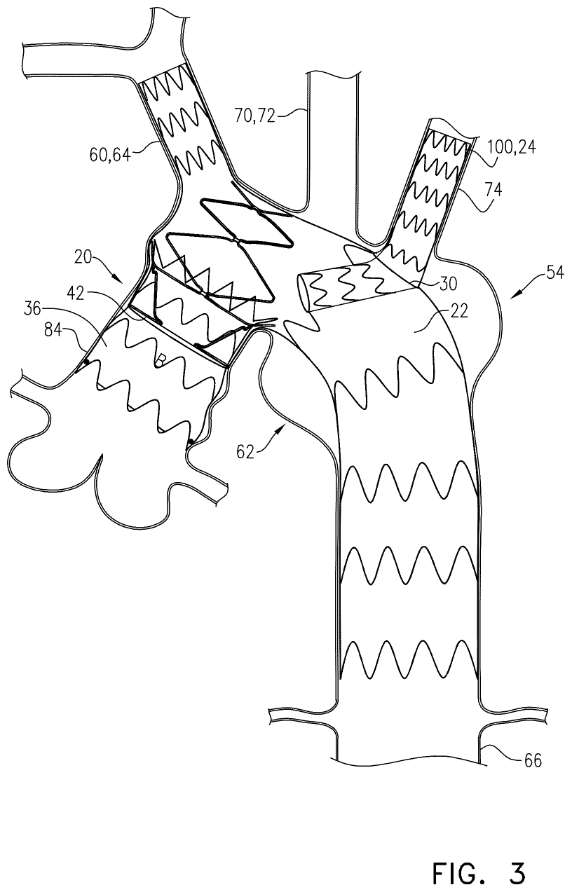

For some applications, branching stent-graft 24 is configured to be positioned partially in a second branch 70 of aortic arch 62 and partially within main stent-graft 22 within aortic arch 62. For example, second branch 70 may be LCCA 72 (as shown) or LSA 74 (as shown in FIG. 3, described hereinbelow).

For some applications, vascular implant assembly 20 further comprises a branching extension stent-graft 36. Branching extension stent-graft 36 is shown in a radially-expanded deployment state in FIGS. 1A-B and 1C-D. Branching extension stent-graft 36 typically comprises a branching-extension-stent-graft stent member 40 and a generally tubular branching-extension-stent-graft fluid flow guide 38. Branching-extension-stent-graft fluid flow guide 38 is securely attached to branching-extension-stent-graft stent member 40 so as to cover at least a portion of branching-extension-stent-graft stent member 40. For these applications, main-stent-graft fluid flow guide 28 is shaped so as to define an extension lateral opening 42, and branching extension stent-graft 36 is configured to be disposed through extension lateral opening 42 so as to form a blood-tight seal between main-stent-graft fluid flow guide 28 and branching-extension-stent-graft fluid flow guide 38, such as shown in FIGS. 1B and 1D. Optionally, main-stent-graft fluid flow guide 28 may implement any of the features of the lateral tube described in U.S. Pat. No. 10,485,684 to Marmur et al., which is incorporated herein by reference.

Typically, the flexible stent members described herein are self-expandable (and are biased toward a radially-expanded deployment state), though they may alternatively be balloon-expandable. Typically, each of the flexible stent members comprises one or more stent struts that may or may not be interconnected, and may or may not be arranged as rings that are optionally axially separate from one another. In configurations in which the stent struts are not interconnected, they are held together by the generally tubular fluid flow guide. For example, the flexible stent members may comprise a metal, such as a superelastic metal alloy, a shape memory metallic alloy, and/or Nitinol; alternatively, the metal may comprise stainless steel.

Each of the generally tubular fluid flow guides may comprise at least one piece of biologically-compatible substantially blood-impervious fabric. The fabric may comprise, for example, a polyester, a polyethylene (e.g., a poly-ethylene-terephthalate), a polymeric film material (e.g., polytetrafluoroethylene (PTFE), polypropylene, polyethylene, high-density polyethylene, polyurethane, polyolefins, ePTFE), a polymeric textile material (e.g., woven polyethylene terephthalate (PET)), natural tissue graft (e.g., saphenous vein or collagen), or a combination thereof.

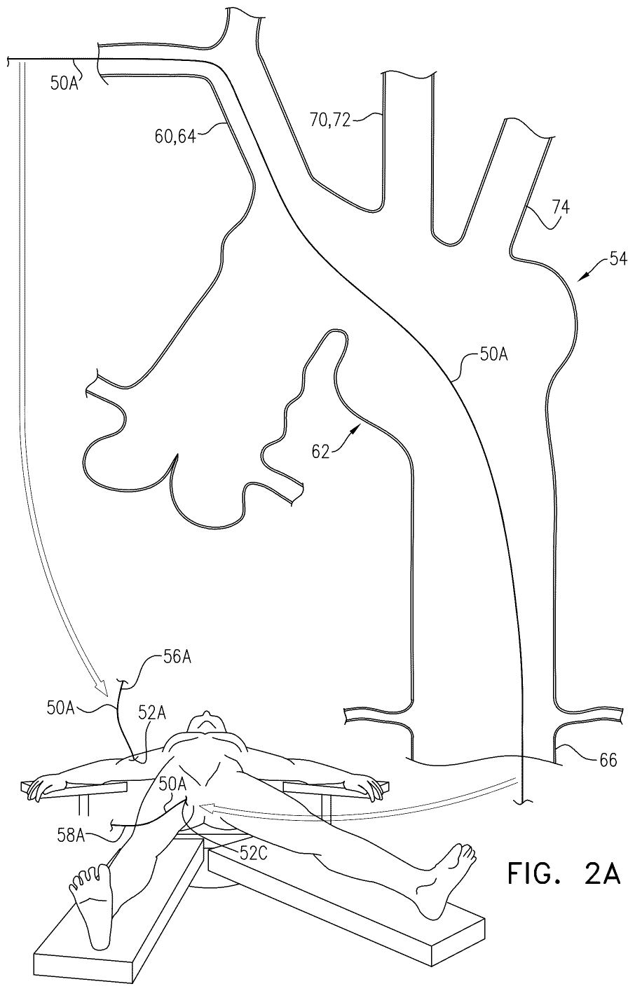

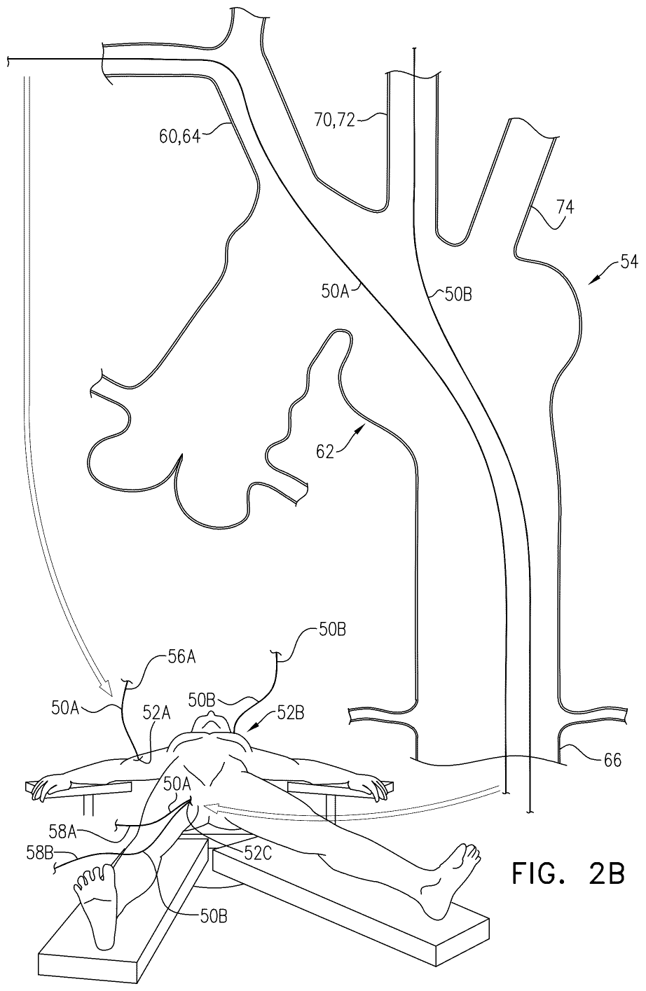

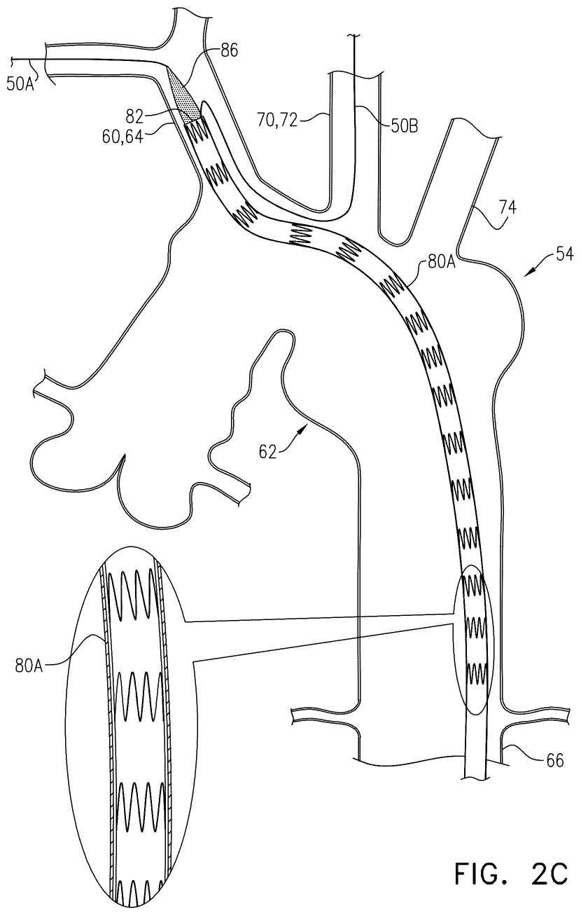

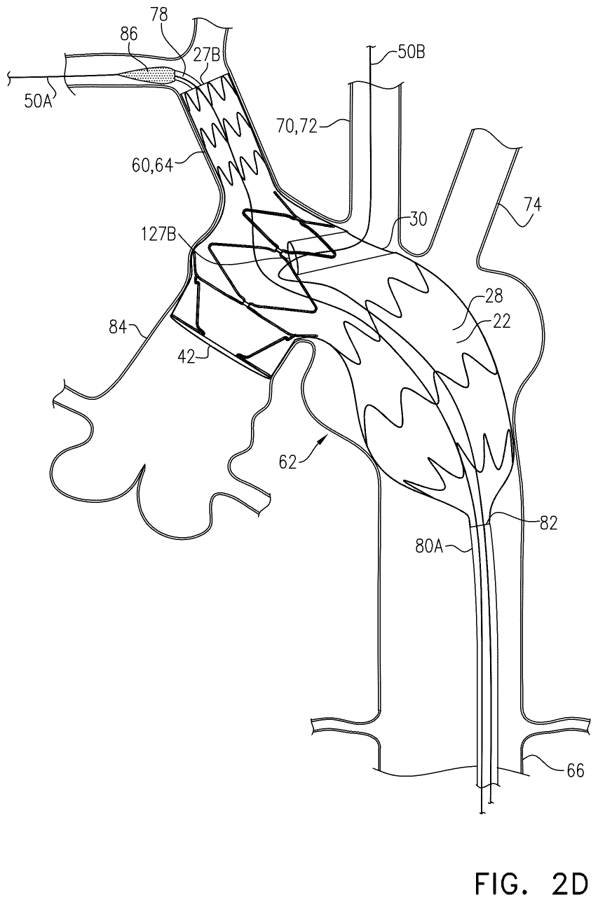

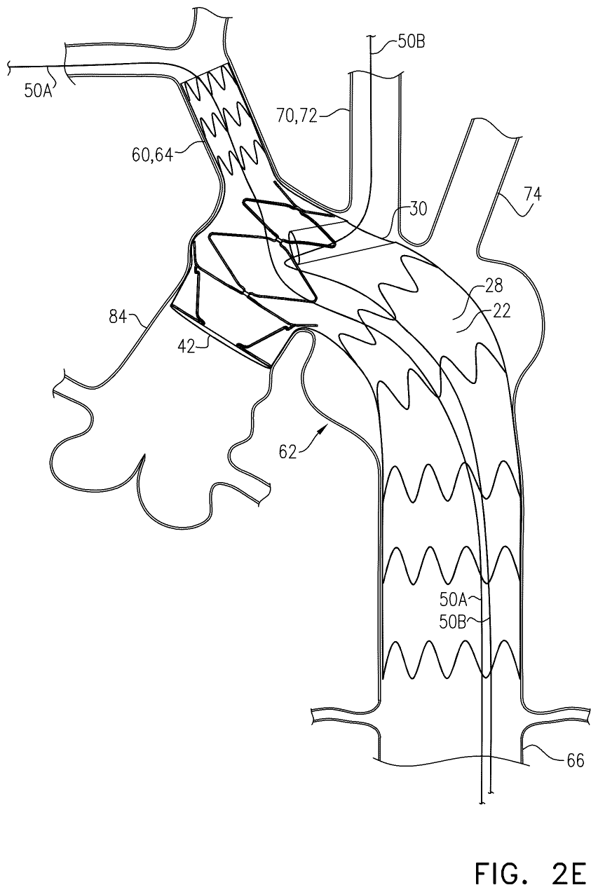

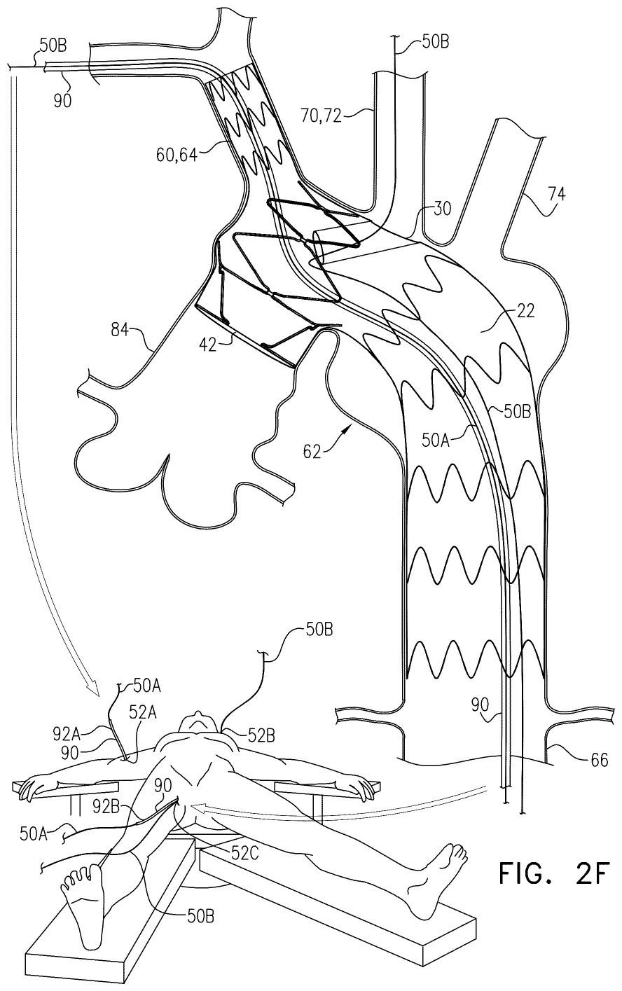

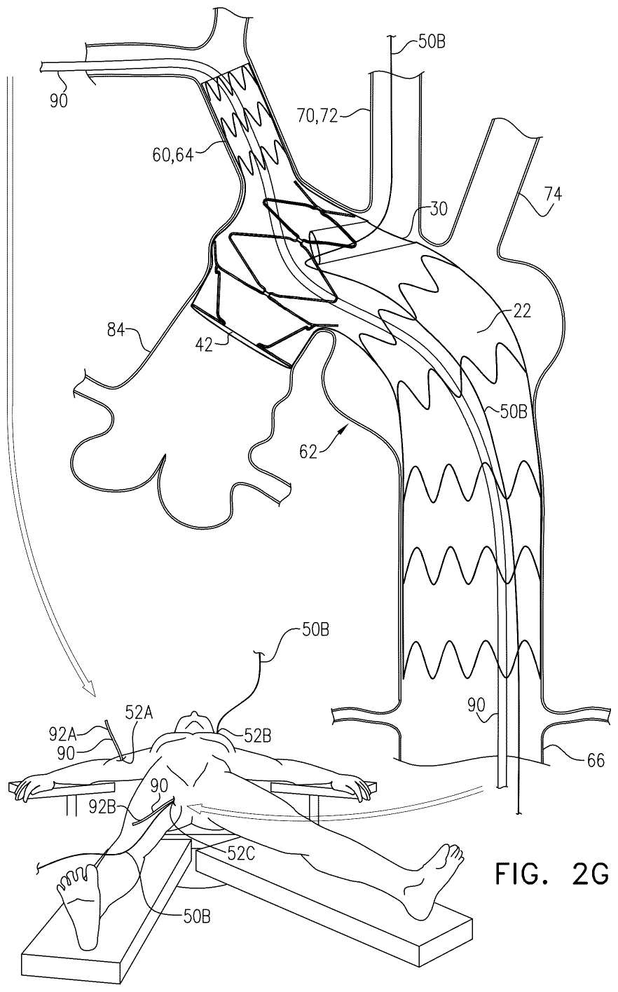

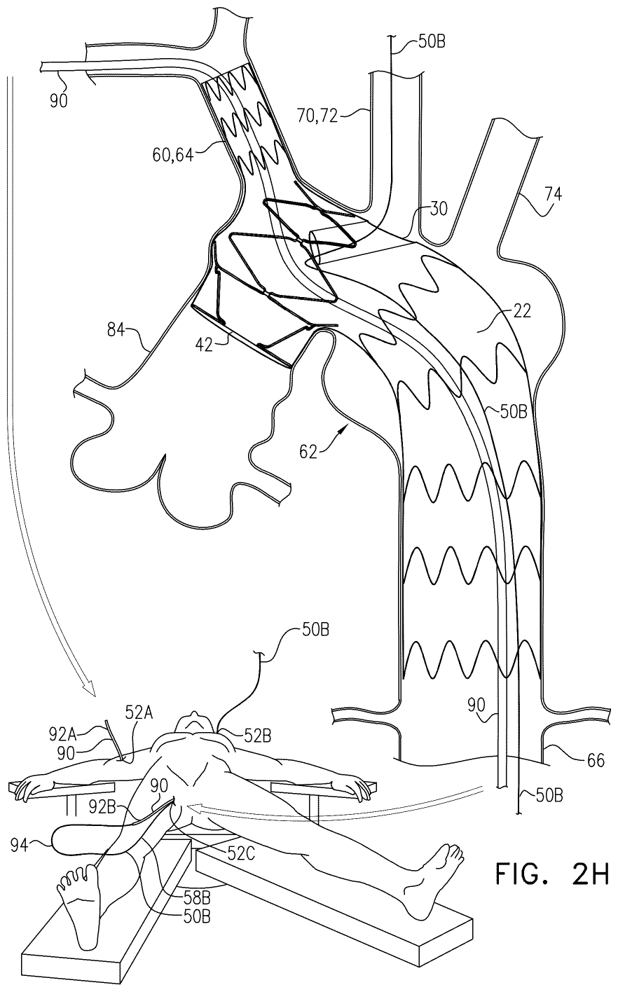

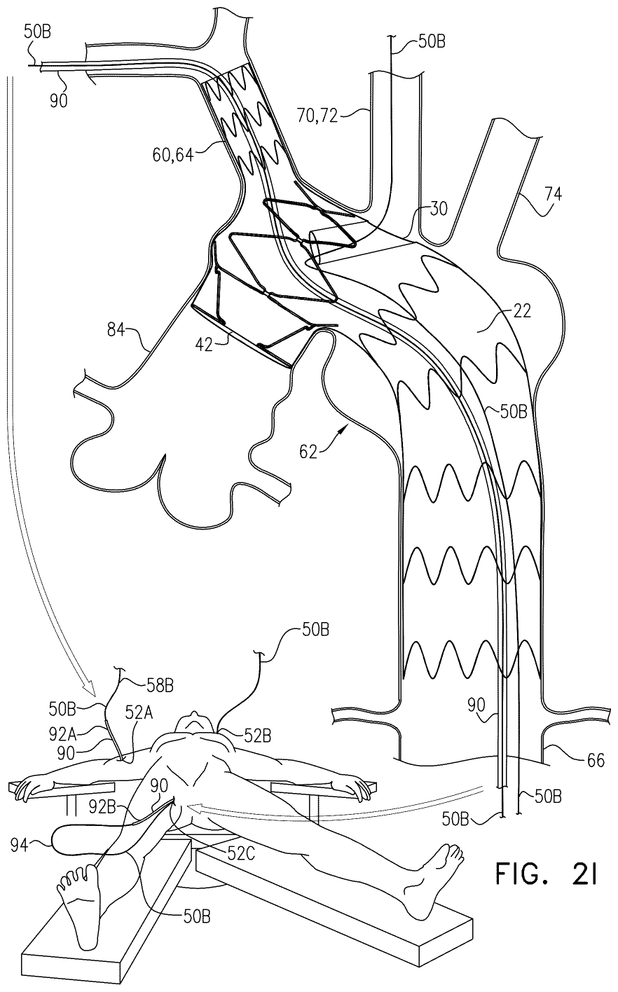

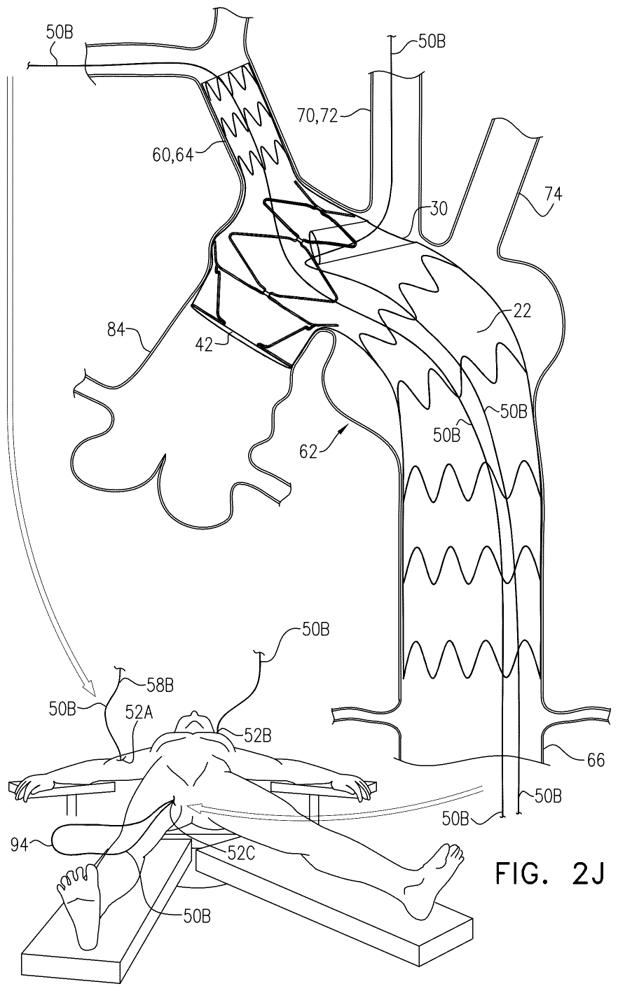

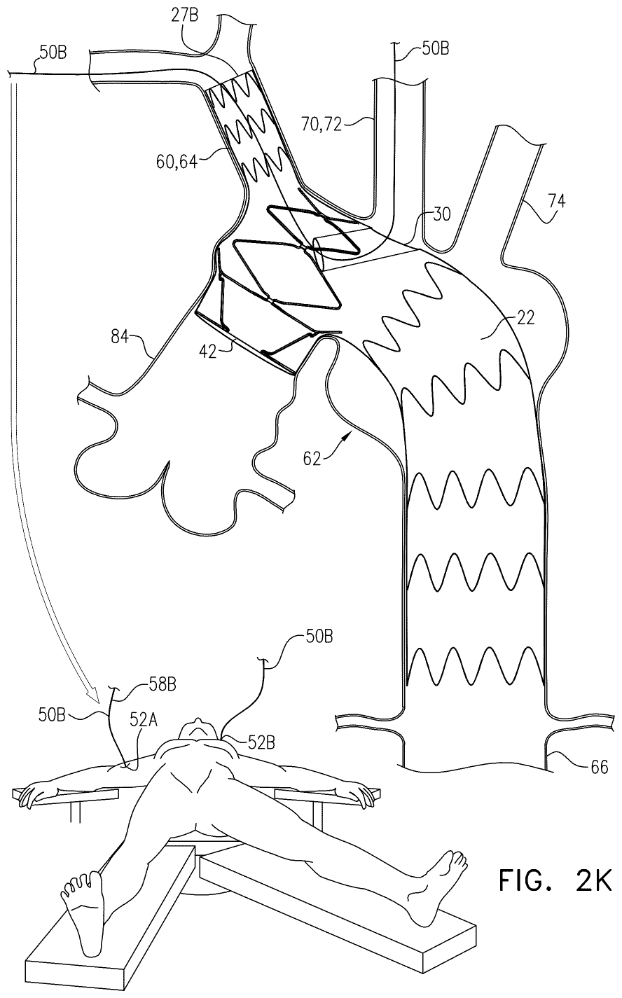

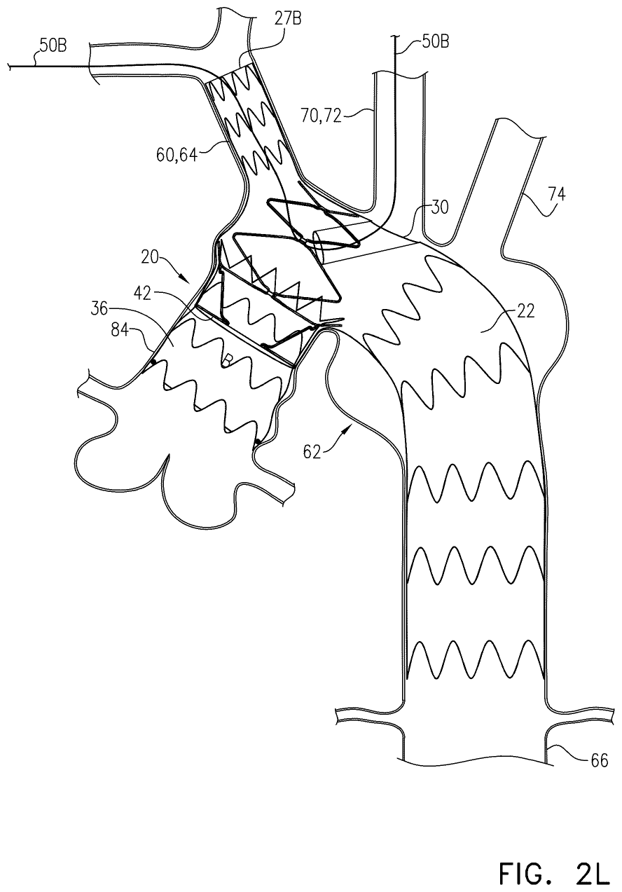

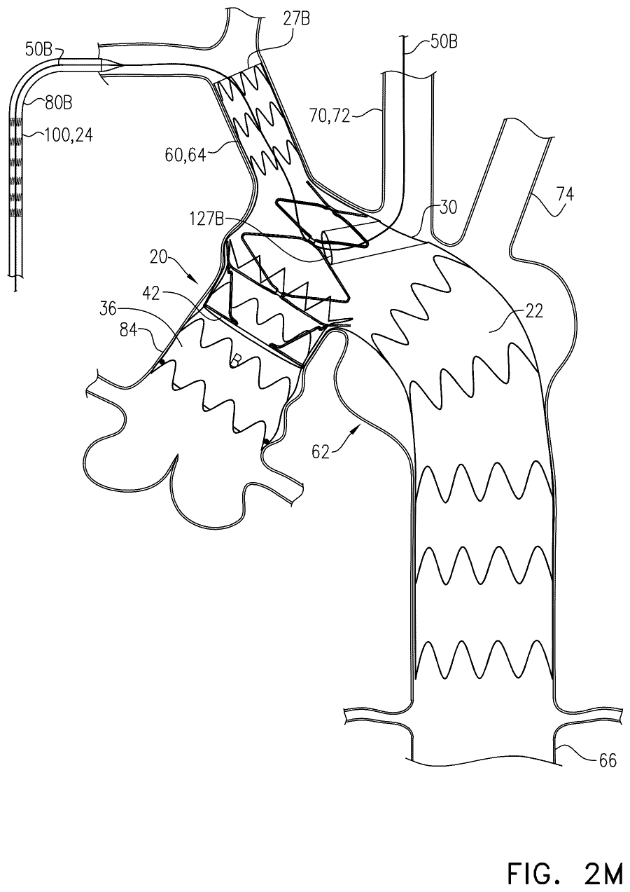

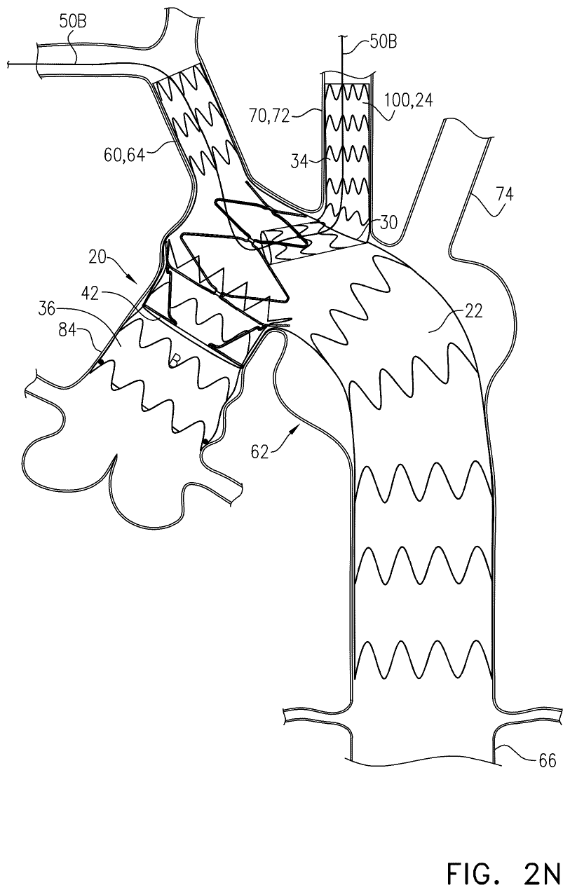

FIGS. 2A-O are schematic illustrations of several stages of a method for deploying one or more vascular implants, in accordance with an application of the present invention. For some applications, the method is used to treat an aortic arch 62 that suffers from acute aortic syndrome, such an aneurysm, a dissection, a penetrating aortic ulcer (PAU), and/or an intramural hematoma (IMH). Although the method is illustrated as comprising deploying main stent-graft 22 and branching stent-graft 24 of the configuration of vascular implant assembly 20 described hereinabove with reference to FIGS. 1A-B, the method may alternatively be used for deploying the configuration of vascular implant assembly 20 described hereinabove with reference to FIGS. 1C-D, or for deploying one or more other vascular implants, such as one or more uncovered stents, one or more balloon catheters, or one or more stent-grafts other than those of vascular implant assembly 20. The method may include all or only a subset of the steps described hereinbelow, and/or additional steps, including but not limited to those described hereinbelow.

As shown in FIG. 2A, a primary guidewire 50A is deployed endovascularly (typically percutaneously) between a first vascular access site 52A and a third vascular access site 52C of vasculature 54 of a patient, such that (a) a first end portion 56A of primary guidewire 50A passes out of vasculature 54 at first vascular access site 52A, and (b) a second end portion 58A of primary guidewire 50A, opposite first end portion 56A of primary guidewire 50A, passes out of vasculature 54 at third vascular access site 52C. Primary guidewire 50A may be deployed using techniques known in the art for deploying through-and-through guidewires. Primary guidewire 50A may be introduced into and advanced within the vasculature in either direction (i.e., from third vascular access site 52C to first vascular access site 52A or vice versa).

Typically, first vascular access site 52A is downstream of first branch 60 of aortic arch 62. For example, first vascular access site 52A may be on a right axillary artery (RAA) in applications in which first branch 60 is BCT 64, such as shown.

Typically, third vascular access site 52C is downstream of descending aorta 66. For example, third vascular access site 52C may be on a right femoral artery, as illustrated, or a right iliac artery (approach not shown).

As shown in FIG. 2B, a secondary guidewire 50B is deployed between a second vascular access site 52B and third vascular access site 52C, such that (a) a first end portion 56B of secondary guidewire 50B passes out of vasculature 54 at second vascular access site 52B, and (b) a second end portion 58B of secondary guidewire 50B, opposite first end portion 56B of secondary guidewire 50B, passes out of vasculature 54 at third vascular access site 52C. Secondary guidewire 50B may be deployed using techniques known in the art for deploying through-and-through guidewires. Secondary guidewire 50B may be introduced into and advanced within the vasculature in either direction (i.e., from third vascular access site 52C to second vascular access site 52B to or vice versa).

Typically, second vascular access site 52B is downstream of second branch 70 of aortic arch 62. For example, second vascular access site 52B may be directly on LCCA 72 in applications in which second branch 70 is LCCA 72, or on LSA 74 or one of its downstream branches (e.g., the left axillary or the left brachial) in applications in which second branch 70 is LSA 74 (such as shown in FIG. 3, described hereinbelow).

For example, secondary guidewire 50B may be introduced through a catheter deployed between second vascular access site 52B (e.g., LCCA 72 or LSA 74) and third vascular access site 52C. Alternatively, secondary guidewire 50B may be introduced using an introducer or access sheath (with or without a catheter) via either second vascular access site 52B or third vascular access site 52C, and then snared to bring the end portion of the secondary guidewire 50B to its target location in vasculature 54.

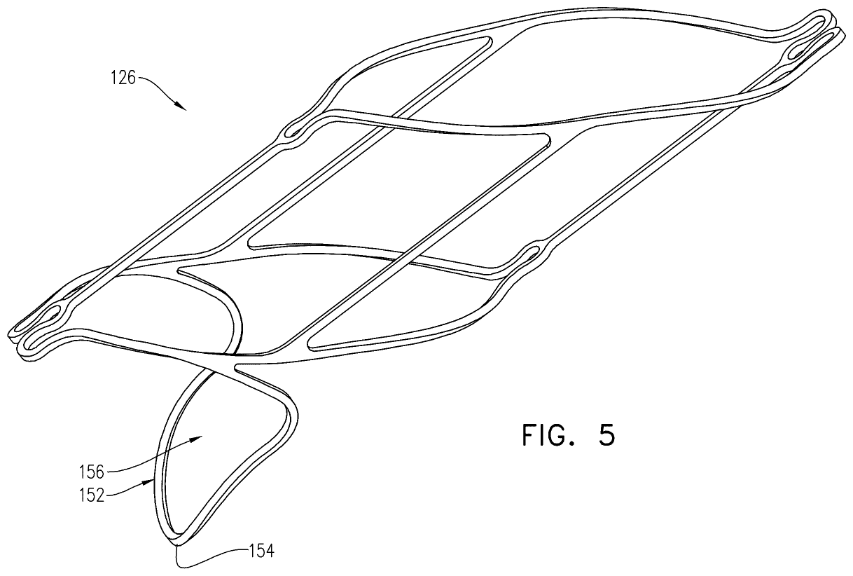

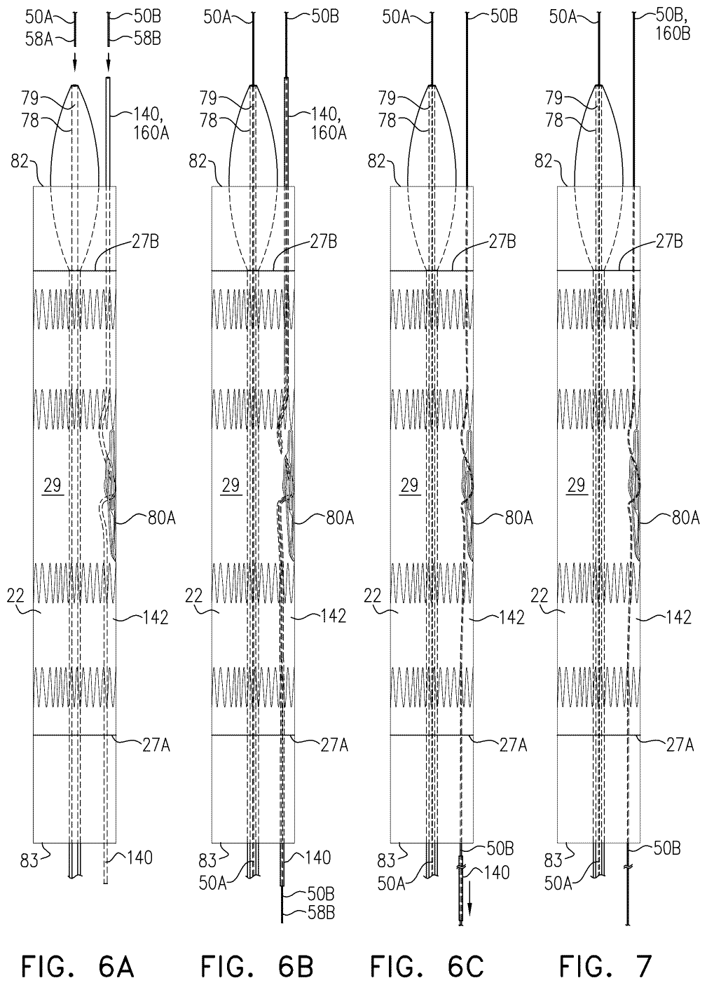

For some applications, the steps of the method described hereinbelow with reference to FIGS. 6A-C are performed (a) after deploying primary guidewire 50A and secondary guidewire 50B, such as described hereinabove with reference to FIGS. 2A and 2B, respectively, and (b) before deploying main stent-graft 22 over primary guidewire 50A, such as described hereinbelow with reference to FIGS. 2C-E.

As shown in FIGS. 2C-E, main stent-graft 22 is deployed over primary guidewire 50A at least partially in aortic arch 62, such that main-fluid-flow guide lateral opening 30 oriented toward second branch 70 (as can better be seen FIGS. 2D-E). For example, a delivery system may be provided that comprises a main delivery catheter 80A and a main inner shaft 78, which is shaped so as to define a guidewire longitudinal lumen 79 through which primary guidewire 50A passes (shown in FIGS. 6A-C and 7, described hereinbelow). Typically, the delivery system further comprises a nosecone 86, as is known in the catheter art. For some applications, proper rotational alignment and/or axial orientation of main-fluid-flow guide lateral opening 30 is achieved using fluoroscopy. For example, main stent-graft 22 may comprise one or more radiopaque markers in a vicinity (e.g., on a periphery of) main-fluid-flow guide lateral opening 30.

Typically, main stent-graft 22 is deployed by:
introducing main delivery catheter 80A into vasculature 54 through third vascular access site 52C and advancing main delivery catheter 80A over primary guidewire 50A and secondary guidewire 50B while main stent-graft 22 is disposed within a portion of main delivery catheter 80A, constrained in a radially-compressed delivery state by main delivery catheter 80A (typically disposed radially between main delivery catheter 80A and main inner shaft 78 (labeled in FIGS. 2D, 6A-C, and 7), extending proximally from distal catheter end 82), as shown in FIG. 2C, and
releasing main stent-graft 22 from the portion of main delivery catheter 80A, such that main stent-graft 22 transitions to a radially-expanded deployment state at least partially in aortic arch 62, such as shown in FIGS. 2D-E; main stent-graft 22 is typically released from main delivery catheter 80A by proximally withdrawing the main delivery catheter, as shown in FIG. 2D.

In configurations in which secondary guidewire 50B is removably pre-positioned passing sequentially inter alia through proximal catheter end 83 and out of distal catheter end 82, such as shown in FIGS. 4D-F and 7, during the advancing of main delivery catheter 80A over primary guidewire 50A and secondary guidewire 50B, main stent-graft may slide distally over secondary guidewire 50B. Alternatively or additionally, main stent-graft 22 may remain axially stationary with respect to secondary guidewire 50B, in which case secondary guidewire 50B may move with the delivery system and may move distally out of a secondary access sheath at second vascular access site 52B (the secondary access sheath is used to introduce secondary guidewire 50B into the vasculature, as known in the guidewire art).

FIG. 2D shows main stent-graft 22 partially released from the main delivery catheter, while FIG. 2E shows the main stent-graft fully released from the main delivery catheter.

For some applications, such as shown in FIGS. 2C-E, a distal-most portion of main stent-graft 22 is deployed in first branch 60 of aortic arch 62. For example, first branch 60 may be BCT 64, such as shown. Alternatively, a distal-most portion of main stent-graft 22 is deployed in aortic arch 62 (configuration not shown).

Typically, as main delivery catheter 80A is advanced over primary guidewire 50A and secondary guidewire 50B, as shown in FIG. 2C, both primary guidewire 50A and secondary guidewire 50B pass through a distal catheter end 82 of main delivery catheter 80A. Typically, secondary guidewire 50B passes:
from distal catheter end 82 of main delivery catheter 80A,
to within a distal portion of main delivery catheter 80A outside main stent-graft 22,
through main-fluid-flow guide lateral opening 30,
to within main stent-graft 22, and
out of proximal main-fluid-flow guide end opening 27A within main delivery catheter 80A,
   for example as described in detail hereinbelow with reference to FIGS. 4A-C, 6A-C, and 7. Typically, primary guidewire 50A and secondary guidewire 50B additionally pass through main delivery catheter 80A until outside of the body of the patient (not shown).

As described above, in some configurations main-stent-graft fluid flow guide 28 is shaped so as to define extension lateral opening 42. In some of these configurations, main stent-graft 22 is deployed over primary guidewire 50A at least partially in aortic arch 62, such that extension lateral opening 42 is disposed in aortic arch 62 facing upstream, oriented toward an ascending aorta 84, in a vicinity of the bifurcation of aortic arch 62 and BCT 64, such as shown in FIGS. 2D-E.

As shown in FIG. 2F, a through-and-through catheter 90 is deployed over primary guidewire 50A between first vascular access site 52A and third vascular access site 52C, such that (a) a first end portion 92A of through-and-through catheter 90 passes out of vasculature 54 at first vascular access site 52A, and (b) a second end portion 92B of through-and-through catheter 90, opposite first end portion 92A, passes out of vasculature 54 at third vascular access site 52C.

As shown in FIG. 2G, primary guidewire 50A is removed (withdrawn) from through-and-through catheter 90, leaving through-and-through catheter 90 in place within vasculature 54.

As shown in FIG. 2H, second end portion 58B of secondary guidewire 50B is reinserted into vasculature 54 through third vascular access site 52C via through-and-through catheter 90, so as to form second end portion 58B of secondary guidewire 50B into a loop 94 outside a body of the patient.

As shown in FIG. 2I, second end portion 58B of secondary guidewire 50B is advanced through through-and-through catheter 90 and out of first vascular access site 52A.

As shown in FIG. 2J, through-and-through catheter 90 is removed from vasculature 54 via first vascular access site 52A.

As shown in the transition between FIG. 2J and FIG. 2K, loop 94 is drawn into vasculature 54 via third vascular access site 52C, typically by pulling on second end portion 58B of secondary guidewire 50B outside of first vascular access site 52A. As a result, secondary guidewire 50B is deployed between first vascular access site 52A and second vascular access site 52B, such that:
secondary guidewire 50B is positioned partially within first branch 60, partially within aortic arch 62, and partially within second branch 70, and
secondary guidewire 50B is positioned passing between distal main-fluid-flow guide end opening 27B and main-fluid-flow guide lateral opening 30, via a portion of a lumen 98 defined by main stent-graft 22.

Optionally, the steps of the method shown in FIGS. 2J and 2K are performed simultaneously, i.e., through-and-through catheter 90 is removed from vasculature 54 as second end portion 58B of secondary guidewire 50B is pulled through first vascular access site 52A.

As shown in FIG. 2L, branching extension stent-graft 36 is deployed through extension lateral opening 42 so as to form a blood-tight seal between main-stent-graft fluid flow guide 28 and branching-extension-stent-graft fluid flow guide 38. Although the deployment of branching extension stent-graft 36 is shown between the steps of FIGS. 2K and 2M, branching extension stent-graft 36 may alternatively be deployed at any point between the steps of FIGS. 2E and 2N.

As shown in FIGS. 2M-N, branching stent-graft 24 is deployed by advancing branching stent-graft 24 over secondary guidewire 50B (a) from first vascular access site 52A to within main stent-graft 22, (b) from within main stent-graft 22, and (c) partially into second branch 70 via main-fluid-flow guide lateral opening 30. As a result, branching stent-graft 24 is partially in second branch 70 external to main-fluid-flow guide lateral opening 30 and partially inside main stent-graft 22, forming the blood-tight seal between main-stent-graft fluid flow guide 28 and branching-stent-graft fluid flow guide 34. For example, the above-described delivery system may further comprise a branching delivery catheter 80B and a branching inner shaft (not shown), which is shaped so as to define a guidewire longitudinal lumen through which secondary guidewire 50B passes.

For some applications, main stent-graft 22 comprises an internal support channel 120, such as described in detail hereinbelow with reference to FIGS. 4A-C, 4D-F, 5, 6A-C, and 7. In these applications, at this stage of deployment a distal channel-fluid-flow guide end opening 127B of internal support channel 120 faces at least partially distally within main fluid flow guide 28 (e.g., partially distally and partially toward a central axis of main fluid flow guide 28, such as shown). In applications in which stent-graft system 110 comprises a secondary guidewire tube 140, such as described hereinbelow with reference to FIGS. 4A-C and 6A-C, this orientation may facilitate inserting secondary guidewire 50B into distal channel-fluid-flow guide end opening 127B, through internal support channel 120, and through main-fluid-flow guide lateral opening 30. After main stent-graft 22 has been transitioned to the radially-expanded deployment state, as shown in the transition between FIGS. 2C and 2D, as described hereinbelow, this orientation may also facilitate advancing branching delivery catheter 80B along this path over secondary guidewire 50B, such as described hereinbelow with reference to FIGS. 2M and 2N.

Typically, branching stent-graft 24 is advanced over secondary guidewire 50B while branching stent-graft 24 is disposed in a radially-compressed delivery state within a portion of branching delivery catheter 80B, such as shown in FIG. 2M. Thereafter, branching stent-graft 24 is released from the portion of branching delivery catheter 80B, such that branching stent-graft 24 transitions to a radially-expanded deployment state at least partially in second branch 70, such as shown in FIG. 2N. Branching stent-graft 24 is typically released from branching delivery catheter 80B by proximally withdrawing the branching delivery catheter.

As shown in FIG. 20, secondary guidewire 50B is removed from vasculature 54 via first vascular access site 52A or second vascular access site 52B, leaving vascular implant assembly 20 (including main stent-graft 22, branching stent-graft 24, and branching extension stent-graft 36) implanted in vasculature 54.

Reference is now made to FIG. 3, which is a schematic illustration of an alternative implantation of vascular implant assembly 20 in vasculature 54, in accordance with an application of the present invention. In this implantation, branching stent-graft 24 is positioned partially in LSA 74. Although this alternative implantation is illustrated using main stent-graft 22 and branching stent-graft 24 of the configuration of vascular implant assembly 20 described hereinabove with reference to FIGS. 1A-B, this alternative implantation may alternatively be used with the configuration of vascular implant assembly 20 described hereinabove with reference to FIGS. 1C-D.

Reference is made to FIGS. 2M-O and 3. In some applications of the present invention, a method is provided that comprises:
endovascularly introducing a vascular implant 100 into vasculature 54 through a vascular access site 52 that is downstream of first branch 60 of aortic arch 62; vascular implant 100 may comprise branching stent-graft 24 or another vascular implant, such as a non-branching stent-graft, an uncovered stent, or a balloon catheter, and/or vascular access site 52 may be first vascular access site 52A or another vascular access site;
thereafter, advancing vascular implant 100 through first branch 60 to aortic arch 62, such as shown in FIG. 2M;
thereafter, advancing vascular implant 100 from aortic arch 62 at least partially into second branch 70 of aortic arch 62; and
thereafter, deploying vascular implant 100 at least partially in second branch 70, such as shown in FIG. 2N.

For some applications in which vascular access site 52 is first vascular access site 52A, the method further comprises deploying a guidewire 50 between first vascular access site 52A and second vascular access site 52B that is downstream of second branch 70. As a result, the guidewire is positioned partially within first branch 60, partially within aortic arch 62, and partially within second branch 70, such as shown in FIG. 2M for secondary guidewire 50B. Advancing vascular implant 100 through first branch 60 to aortic arch 62 and from aortic arch 62 at least partially into second branch 70 comprises:
advancing vascular implant 100 over guidewire 50 through first branch 60 to aortic arch 62; and
thereafter, advancing vascular implant 100 over guidewire 50 from aortic arch 62 at least partially into second branch 70.

For some of these applications in which guidewire 50 is deployed between first vascular access site 52A and second vascular access site 52B, vascular implant 100 is endovascularly introduced into vasculature 54 while vascular implant 100 is disposed in a radially-compressed delivery state within a portion of a delivery catheter 80, such as branching delivery catheter 80B, such as shown in FIG. 2M for branching stent-graft 24. Vascular implant 100 is advanced through first branch 60 to aortic arch 62 and from aortic arch 62 at least partially into second branch 70, by advancing the portion of delivery catheter 80 through first branch 60 to aortic arch 62 and from aortic arch 62 at least partially into second branch 70. Vascular implant 100 is deployed at least partially in second branch 70 by releasing vascular implant 100 from the portion of delivery catheter 80, such that vascular implant 100 transitions to a radially-expanded deployment state at least partially in second branch 70.

Reference is still made to FIGS. 2A-O and 3. In some applications of the present invention, a method is provided that comprises:
deploying guidewire 50 between (i) first vascular access site 52A that is downstream of first branch 60 of aortic arch 62 and (ii) second vascular access site 52B that is downstream of second branch 70 of aortic arch 62, such that guidewire 50 is positioned partially within first branch 60, partially within aortic arch 62, and partially within second branch 70, such as shown in FIGS. 2K-L for secondary guidewire 50B;
advancing vascular implant 100 over guidewire 50, such as shown in FIG. 2M for branching stent-graft 24; and
deploying vascular implant 100 within vasculature of the patient, such as shown in FIG. 2N for branching stent-graft 24.

Vascular implant 100 may comprise branching stent-graft 24 or another vascular implant, such as a non-branching stent-graft, an uncovered stent, or a balloon catheter.

For some applications, deploying guidewire 50 between first vascular access site 52A and second vascular access site 52B, such that guidewire 50 is positioned partially within first branch 60, partially within aortic arch 62, and partially within second branch 70, comprises:
deploying guidewire 50 between (i) second vascular access site 52B and (ii) third vascular access site 52C that is downstream of descending aorta 66, such as shown in FIG. 2N for secondary guidewire 50B, such that:
   guidewire 50 is positioned partially within second branch 70, partially within aortic arch 62, and partially within descending aorta 66,
   a first end portion 56 of guidewire 50 passes out of vasculature 54 at second vascular access site 52B, and
   a second end portion 58 of guidewire 50, opposite first end portion 56, passes out of vasculature 54 at third vascular access site 52C;
forming second end portion 58 of guidewire 50 into loop 94 outside a body of the patient by reinserting the second end portion of guidewire 50 into vasculature 54 through third vascular access site 52C, such as shown in FIG. 2H for secondary guidewire 50B; and
thereafter, advancing second end portion 58 of guidewire 50 (a) through descending aorta 66, (b) through a portion of aortic arch 62, (c) through at least a portion of first branch 60, and (d) out of first vascular access site 52A, such that loop 94 is drawn into vasculature 54 via third vascular access site 52C, such as shown in FIGS. 2I-K for secondary guidewire 50B.

For some applications in which guidewire 50 is secondary guidewire 50B, secondary guidewire 50B is deployed between first vascular access site 52A second vascular access site 52B by deploying primary guidewire 50A between first vascular access site 52A and third vascular access site 52C, such that (a) first end portion 56A of primary guidewire 50A passes out of vasculature 54 at first vascular access site 52A, and (b) second end portion 58A of primary guidewire 50A, opposite first end portion 56A of primary guidewire 50A, passes out of vasculature 54 at third vascular access site 52C, such as shown in FIG. 2B. Optionally, some or all of the steps of the method described hereinabove with reference to FIGS. 2C-J are additionally performed.

For some applications, vascular implant 100 is advanced over guidewire 50 while vascular implant 100 is disposed in a radially-compressed delivery state within a portion of delivery catheter 80, such as branching delivery catheter 80B, such as shown in FIG. 2M for branching stent-graft 24. Vascular implant 100 is deployed within vasculature 54 by releasing vascular implant 100 from the portion of delivery catheter 80, such that vascular implant 100 transitions to a radially-expanded deployment state within vasculature 54, such as shown in FIG. 2N for branching stent-graft 24.

For some applications, vascular implant 100 is advanced over guidewire 50 from first vascular access site 52A to aortic arch 62, and from aortic arch 62 at least partially into second branch 70, such as shown in FIGS. 2M-N for branching stent-graft 24. Optionally, before deploying branching stent-graft 24 within vasculature 54, main stent-graft 22 is deployed at least partially in aortic arch 62, such that main-fluid-flow guide lateral opening 30 is oriented toward second branch 70, such as described hereinabove with reference to FIGS. 2M-N.

Reference is still made to FIGS. 2A-O and 3. Optionally, the deployment techniques described herein are implemented in combination with one or more of the deployment techniques described in U.S. Pat. No. 8,945,203 to Shalev et al., which is incorporated herein by reference.

Reference is again made to FIG. 1A and is also made to FIGS. 4A-C, which are side-view schematic illustrations of internal support channel 120 of main stent-graft 22 in an expanded state, a partially-collapsed state, and a collapsed state, respectively, in accordance with an application of the present invention. For example, internal support channel 120 may assume the partially-collapsed state during a transition from the expanded state to the collapsed state during manufacture, and/or during a transition from the collapsed state to the expanded state during deployment, such as described hereinbelow. Although FIGS. 4A-C illustrate main stent-graft 22 and branching stent-graft 24 of the configuration of vascular implant assembly 20 described hereinabove with reference to FIGS. 1A-B, many of the features described with reference to FIGS. 4A-C are also applicable to the configuration of vascular implant assembly 20 described hereinabove with reference to FIGS. 1C-D, mutatis mutandis.

FIGS. 4A-C also show a portion of a stent-graft system 110, which comprises a main stent-graft, such as, by way of example and not limitation, main stent-graft 22, described hereinabove with reference to FIGS. 1A-3.

Reference is also made to FIG. 5, which is a schematic illustration of a flexible channel stent member 126 of internal support channel 120 in the expanded state, from a partially distal and partially side view, in accordance with an application of the present invention. For clarity of illustration, channel fluid flow guide 128 is not shown in FIG. 5, although it is actually present. Flexible channel stent member 126 shown in FIG. 5 may be implemented with either the configuration of vascular implant assembly 20 described hereinabove with reference to FIGS. 1A-B or the configuration of vascular implant assembly 20 described hereinabove with reference to FIGS. 1C-D.

In these configurations, internal support channel 120 is configured to assume expanded and collapsed states, such as shown in FIGS. 4A-C. Internal support channel 120 is disposed within main fluid flow guide 28. Internal support channel 120 comprises a generally tubular channel fluid flow guide 128, which is shaped so as to define:
proximal and distal channel-fluid-flow end openings 127A and 127B, and
when internal support channel 120 is in the expanded state, as shown in FIG. 4A, a channel lumen 129 between proximal and distal channel-fluid-flow end openings 127A and 127B.

Proximal channel-fluid-flow end opening 127A is sealingly coupled to a perimeter of main-fluid-flow guide lateral opening 30, typically around the entire perimeter of the lateral opening, such that channel lumen 129 is in fluid communication with outside main fluid flow guide 28 via main-fluid-flow guide lateral opening 30 when internal support channel 120 is in the expanded state.

For some applications, such as shown in FIGS. 1C-D, an external surface 131 (labeled in FIG. 1C and FIG. 4A) of internal support channel 120 is coupled to an internal surface 133 of main fluid flow guide 28 such that internal support channel 120 runs alongside the internal surface of main fluid flow guide 28 distally from main-fluid-flow guide lateral opening 30. Typically, in these applications, tubular channel fluid flow guide 128 is coupled to internal surface 133 of main fluid flow guide 28, such as by being coupled (e.g., by stitching 137) to one or more struts of main-stent-graft stent member 26. (Stitching 137 is in addition to any other stitching (shown by way of example as smaller than stitching 137) that couples main fluid flow guide 28 itself to the struts of main-stent-graft stent member 26.) As shown in FIG. 4C, when internal support channel 120 is in the collapsed state, distal channel-fluid-flow guide end opening 127B faces radially inward (i.e., toward a central longitudinal axis 135 of main fluid flow guide 28).

As shown in FIG. 4A, when internal support channel 120 is in the expanded state and main stent-graft 22 is in the radially-expanded deployment state, distal channel-fluid-flow guide end opening 127B faces at least partially distally within main fluid flow guide 28 (optionally angled with respect to central longitudinal axis 135 of main fluid flow guide 28, such as shown). Typically, when internal support channel 120 is in the expanded state and main stent-graft 22 is in the radially-expanded deployment state, distal channel-fluid-flow guide end opening 127B is disposed distal to main-fluid-flow guide lateral opening 30.

In some applications of the present invention, stent-graft system 110 further comprises a secondary guidewire tube 140, which is removably positioned passing sequentially through:
typically, proximal catheter end 83,
proximal main-fluid-flow guide end opening 27A,
inside a longitudinal portion 142 of main fluid flow lumen 29,
distal channel-fluid-flow guide end opening 127B,
main-fluid-flow guide lateral opening 30,
along the outside of main fluid flow lumen 29, and
typically, out of (e.g., 0.5-2 cm, e.g., about 1 cm out of), or terminating at the same level as, distal catheter end 82.

In this configuration, secondary guidewire tube 140 is an implementation of an elongate member 160A, which is removably positioned passing sequentially through (a) proximal main-fluid-flow guide end opening 27A, (b) longitudinal portion 142 of main fluid flow lumen 29, (c) distal channel-fluid-flow guide end opening 127B, and (d) main-fluid-flow guide lateral opening 30 to outside main fluid flow lumen 29.

Secondary guidewire tube 140 is shaped so as to define a secondary-guidewire-tube lumen for insertion therethrough of the secondary guidewire 50B, such as described hereinbelow with reference to FIG. 6B.

In this configuration, secondary guidewire 50B typically has a diameter of 0.012″-0.016″ (0.305-0.406 mm), such as 0.014″ (0.356 mm).

In this configuration, internal support channel 120 is configured to automatically transition to the expanded state when:
not constrained in the collapsed state by main stent-graft 22, such as when at least a longitudinal portion of main stent-graft 22 alongside internal support channel 120 is in the radially-expanded deployment state, such as shown in FIG. 2D, or is transitioning to the radially-expanded deployment state as internal support channel 120 automatically transitions to the expanded state, and
not constrained by secondary guidewire tube 140, such as (i) after removal of secondary guidewire tube 140, such as described hereinbelow with reference to FIG. 6C, or (ii) if secondary guidewire tube 140 is allowed to pass loosely through internal support channel 120 (i.e., secondary guidewire tube 140 is not tensed sufficiently to constrain internal support channel 120 in the collapsed state).

For some applications, main stent-graft 22 is configured such that secondary guidewire tube 140 is removable from internal support channel 120 while main stent-graft is in the radially-compressed delivery state, such as described hereinbelow with reference to FIG. 6C. In these applications, internal support channel 120 becomes not constrained by secondary guidewire tube 140 before becoming not constrained by main stent-graft 22.

Alternatively or additionally, for some applications, main stent-graft 22 is configured such that secondary guidewire tube 140 is removable from internal support channel 120 while main stent-graft 22 is in the radially-compressed delivery state and secondary guidewire 50B is inserted through the secondary-guidewire-tube lumen, such as described hereinbelow with reference to FIG. 6C.

For some applications, as labeled in FIG. 4C, a longitudinal portion 144 of secondary guidewire tube 140 includes:
a first longitudinal sub-portion 144A extending between proximal main-fluid-flow guide end opening 27A and main-fluid-flow guide lateral opening 30, via distal channel-fluid-flow guide end opening 127B, and
a second longitudinal sub-portion 144B extending out of main-fluid-flow guide lateral opening 30, outside main fluid flow lumen 29, and having a length of 1 cm, measured from main-fluid-flow guide lateral opening 30 to a distal end 146 of second longitudinal sub-portion 144B.

Typically, secondary guidewire tube 140 further includes a distal portion extending distally from distal end 146 of second longitudinal sub-portion 144B, and out of distal catheter end 82 of main delivery catheter 80A.

For some applications, such as shown in FIG. 4C, when distal end 146 of second longitudinal sub-portion 144B is held in contact with an external surface of main stent-graft 22 (such as by main delivery catheter 80A) while main stent-graft 22 is in the radially-compressed delivery state, longitudinal portion 144 of secondary guidewire tube 140:
is almost straight, i.e., has a smallest radius of curvature, at any location along longitudinal portion 144, that is at least 0.15 cm, such as at least 0.3 cm, at least 0.5 cm, at least 1 cm, at least 2 cm, at least 3 cm, or at least 5 cm,
is not folded,
is not kinked,
is neither folded nor kinked, and/or
defines a directional path 148 from proximal main-fluid-flow guide end opening 27A to distal end 146 of second longitudinal sub-portion 144B, and directional path 148 does not include any proximally-directed portions.

The above-mentioned properties may enable insertion of secondary guidewire 50B through the secondary-guidewire-tube lumen while main stent-graft 22 is in the radially-compressed delivery state, such as described hereinbelow with reference to FIG. 6B.

For some applications, such as shown in FIG. 4C, internal support channel 120 is configured such that when internal support channel 120 is in the collapsed state, a proximal-most point 150A of distal channel-fluid-flow guide end opening 127B is disposed proximal of a distal-most point 150B of main-fluid-flow guide lateral opening 30. Secondary guidewire tube 140 passes through a gap 151 defined between proximal-most point 150A and distal-most point 150B. (Gap 151 is defined by axially overlapping portions of distal channel-fluid-flow guide end opening 127B and main-fluid-flow guide lateral opening 30.)

For some applications, during loading of main stent-graft 22 into main delivery catheter 80A prior to introduction into vasculature 54, tongue-shaped stent strut 152 is folded proximally back on itself (as illustrated in the transition between FIG. 4A and FIG. 4C). This folding may be facilitated by:
passing secondary guidewire tube 140 sequentially through proximal main-fluid-flow guide end opening 27A, inside a longitudinal portion 142 of main fluid flow lumen 29, distal channel-fluid-flow guide end opening 127B, main-fluid-flow guide lateral opening 30, and along the outside of main fluid flow lumen 29, as shown in FIG. 4A, and
applying tension to secondary guidewire tube 140 before and during compression of main stent-graft 22 into main delivery catheter 80A; the tension primarily helps fold tongue-shaped stent strut 152 in the right orientation (extending proximally) for compression of internal support channel 120; the tension also constrains internal support channel 120 in the collapsed state.

Reference is now made to FIGS. 4D-F, which are side-view schematic illustrations of internal support channel 120 of main stent-graft 22 in the expanded state, the partially-collapsed state, and the collapsed state, respectively, in accordance with another application of the present invention. In this configuration, unlike the configuration described hereinabove with reference to FIGS. 4A-C, stent-graft system 110 does not comprise secondary guidewire tube 140. Instead, secondary guidewire 50B is removably pre-positioned passing sequentially through:
typically, proximal catheter end 83,
proximal main-fluid-flow guide end opening 27A,
inside longitudinal portion 142 of main fluid flow lumen 29,
distal channel-fluid-flow guide end opening 127B,
main-fluid-flow guide lateral opening 30,
along the outside of main fluid flow lumen 29, and
typically, out of (e.g., 150-500 cm, such as 200-400 cm out of) distal catheter end 82.

In this configuration, secondary guidewire 50B is an implementation of an elongate member 160B, which is removably positioned passing sequentially through (a) proximal main-fluid-flow guide end opening 27A, (b) longitudinal portion 142 of main fluid flow lumen 29, (c) distal channel-fluid-flow guide end opening 127B, and (d) main-fluid-flow guide lateral opening 30 to outside main fluid flow lumen 29.

In this configuration, secondary guidewire 50B typically has a diameter of 0.016″-0.02″ (0.406-0.508 mm), such as 0.018″ (0.457 mm).

In this configuration, internal support channel 120 is configured to automatically transition to the expanded state when:
not constrained in the collapsed state by main stent-graft 22, such as when at least a longitudinal portion of main stent-graft 22 alongside internal support channel 120 is in the radially-expanded deployment state, such as shown in FIG. 2D, or is transitioning to the radially-expanded deployment state as internal support channel 120 automatically transitions to the expanded state, and
not constrained by secondary guidewire 50B. As shown in FIG. 2D, secondary guidewire 50B does not constrain internal support channel 120, even though secondary guidewire 50B passes loosely through internal support channel 120. (Typically, secondary guidewire 50B only constrains internal support channel 120 during loading of main stent-graft 22 into main delivery catheter 80A, as described below.)

For some applications, as labeled in FIG. 4F, a longitudinal portion 144 of secondary guidewire 50B:
a first longitudinal sub-portion 144A extending between proximal main-fluid-flow guide end opening 27A and main-fluid-flow guide lateral opening 30, via distal channel-fluid-flow guide end opening 127B, and
a second longitudinal sub-portion 144B extending out of main-fluid-flow guide lateral opening 30, outside main fluid flow lumen 29, and having a length of 1 cm, measured from main-fluid-flow guide lateral opening 30 to a distal end 146 of second longitudinal sub-portion 144B.

As mentioned above, secondary guidewire 50B further includes a distal portion extending distally from distal end 146 of second longitudinal sub-portion 144B, and out of distal catheter end 82 of main delivery catheter 80A.

For some applications, such as shown in FIG. 4F, when distal end 146 of second longitudinal sub-portion 144B is held in contact with an external surface of main stent-graft 22 (such as by main delivery catheter 80A) while main stent-graft 22 is in the radially-compressed delivery state, longitudinal portion 144 of secondary guidewire 50B:
is almost straight, i.e., has a smallest radius of curvature, at any location along longitudinal portion 144, that is at least 0.15 cm, such as at least 0.3 cm, at least 0.5 cm, at least 1 cm, at least 2 cm, at least 3 cm, or at least 5 cm,
is not folded,
is not kinked,
is neither folded nor kinked, and/or
defines a directional path 148 from proximal main-fluid-flow guide end opening 27A to distal end 146 of second longitudinal sub-portion 144B, and directional path 148 does not include any proximally-directed portions.

The above-mentioned properties may prevent damage (such as kinking) to secondary guidewire 50B while main stent-graft 22 is in the radially-compressed delivery state, such as described hereinabove with reference to FIG. 2C and hereinbelow with reference to FIG. 7.

As described hereinabove with reference to FIG. 4C, and as also shown in FIG. 4F, for some applications, internal support channel 120 is configured such that when internal support channel 120 is in the collapsed state, proximal-most point 150A of distal channel-fluid-flow guide end opening 127B is disposed proximal of distal-most point 150B of main-fluid-flow guide lateral opening 30. In the configuration shown in FIG. 4F, secondary guidewire 50B passes through gap 151 defined between proximal-most point 150A and distal-most point 150B. (Gap 151 is defined by axially overlapping portions of distal channel-fluid-flow guide end opening 127B and main-fluid-flow guide lateral opening 30.)

For some applications, during loading of main stent-graft 22 into main delivery catheter 80A prior to introduction into vasculature 54, tongue-shaped stent strut 152 is folded proximally back on itself (as illustrated in the transition between FIG. 4D and FIG. 4E). This folding may be facilitated by:
passing secondary guidewire 50B sequentially through proximal main-fluid-flow guide end opening 27A, inside longitudinal portion 142 of main fluid flow lumen 29, distal channel-fluid-flow guide end opening 127B, main-fluid-flow guide lateral opening 30, and along the outside of main fluid flow lumen 29, as shown in FIG. 4D, and
applying tension to secondary guidewire 50B before and during compression of main stent-graft 22 into main delivery catheter 80A; the tension primarily helps fold tongue-shaped stent strut 152 in the right orientation (extending proximally) for compression of internal support channel 120; the tension also constrains internal support channel 120 in the collapsed state.

Reference is now made to both FIGS. 4A-C and FIG. 4D-F. For some applications, internal support channel 120 further comprises flexible channel stent member 126 to which channel fluid flow guide 128 is securely attached.

For some of these applications, channel stent member 126 comprises one or more stent struts, which are shaped so as to define a tongue-shaped stent strut 152. Tongue-shaped stent strut 152, typically including a tip 154 thereof, is securely attached to a portion of a perimeter of distal channel-fluid-flow guide end opening 127B, so as to help define distal channel-fluid-flow guide end opening 127B.

Reference is still made to both FIGS. 4A-C and FIG. 4D-F. For some applications, a minimal surface 156 defined by tongue-shaped stent strut 152 faces:
at least partially distally when internal support channel 120 is in the expanded state, as shown in FIGS. 4A and 4D, and
at least partially radially inward (i.e., toward central longitudinal axis 135) when internal support channel 120 is in the collapsed state, as shown in FIGS. 4C and 4F.

As used in the present application, including the claims and Inventive Concepts, the “minimal surface” defined by tongue-shaped stent strut 152 is the mathematical surface bounded by the two sides and tip of tongue-shaped stent strut 152 that locally minimizes its area, in accordance with the definition of “minimal surface” known in the mathematical arts. It will be appreciated that the minimal surface is a mathematical construct, rather than a physical element of internal support channel 120.

Reference is still made to both FIGS. 4A-C and FIG. 4D-F. For some applications, tongue-shaped stent strut 152 is configured such that, during the automatic transition of internal support channel 120 from the collapsed state (shown in FIGS. 4C and 4F) to the expanded state (shown in FIGS. 4A and 4D), tip 154 of tongue-shaped stent strut 152 automatically swings along a curved path 157 from facing proximally to facing less proximally, such as partially distally.

Reference is still made to both FIGS. 4A-C and FIG. 4D-F. For some applications, tongue-shaped stent strut 152 is configured such that, during the automatic transition of internal support channel 120 from the collapsed state (shown in FIGS. 4C and 4F) to the expanded state (shown in FIGS. 4A and 4D), tongue-shaped stent strut 152 automatically swings:
from minimal surface 156 facing at least partially radially inward (i.e., toward central longitudinal axis 135),
to minimal surface 156 facing at least partially distally.

Reference is still made to both FIGS. 4A-C and FIG. 4D-F. For some applications, internal support channel 120 is configured such that, during the automatic transition of internal support channel 120 from the collapsed state (shown in FIGS. 4C and 4F) to the expanded state (shown in FIGS. 4A and 4D), a best-fit plane defined by distal channel-fluid-flow guide end opening 127B automatically swings:
from distal channel-fluid-flow guide end opening 127B facing at least partially radially inward (i.e., toward central longitudinal axis 135),
to distal channel-fluid-flow guide end opening 127B facing at least partially distally.

Reference is now made to FIGS. 6A-C, which are schematic illustrations of several stages of a portion of a method for deploying main stent-graft 22 and branching stent-graft 24, in accordance with an application of the present invention. FIGS. 6A-C show main stent-graft 22 disposed in the radially-compressed delivery state within a portion of main delivery catheter 80A. (For clarity of illustration, main stent-graft 22 is shown slightly expanded in FIGS. 6A-C, even though it is in practice generally more radially-compressed when in this delivery state.)

In some applications, the steps of the method illustrated in FIGS. 6A-C are performed (a) after deploying primary guidewire 50A and secondary guidewire 50B, such as described hereinabove with reference to FIGS. 2A and 2B, respectively, and (b) before deploying main stent-graft 22 over primary guidewire 50A, such as described hereinabove with reference to FIGS. 2C-E.

For some applications, such as shown in FIG. 6A, stent-graft system 110 further comprises main delivery catheter 80A, such as described hereinabove with reference to FIGS. 2C-E. Main stent-graft 22 is removably disposed within main delivery catheter 80A in the radially-compressed delivery state with distal main-fluid-flow guide end opening 27B facing distal catheter end 82 of main delivery catheter 80A, and internal support channel 120 in the collapsed state.

As shown in FIG. 6A, secondary guidewire tube 140 is removably positioned passing sequentially through (a) proximal main-fluid-flow guide end opening 27A, (b) longitudinal portion 142 of main fluid flow lumen 29, (c) distal channel-fluid-flow guide end opening 127B, (d) main-fluid-flow guide lateral opening 30 to outside main fluid flow lumen 29 within main delivery catheter 80A, and (e) distal catheter end 82.

For some applications, as shown in FIG. 6A, longitudinal portion 144 (labeled in FIG. 4C) of secondary guidewire tube 140:
extends between proximal main-fluid-flow guide end opening 27A of the radially-compressed main stent-graft 22 and distal catheter end 82, and is almost straight, i.e., has a smallest radius of curvature, at any location along longitudinal portion 144, that is at least 0.15 cm, such as at least 0.3 cm, at least 0.5 cm, at least 1 cm, at least 2 cm, at least 3 cm, or at least 5 cm,
extends between proximal main-fluid-flow guide end opening 27A of the radially-compressed main stent-graft 22 and distal catheter end 82, and is neither folded nor kinked, and/or
defines a directional path from proximal main-fluid-flow guide end opening 27A of the radially-compressed main stent-graft 22 to distal catheter end 82, and the directional path does not include any proximally-directed portions.

As shown in FIG. 6B, secondary guidewire 50B is inserted through the secondary-guidewire-tube lumen of secondary guidewire tube 140 that is removably positioned passing sequentially through (a) proximal main-fluid-flow guide end opening 27A, (b) longitudinal portion 142 of main fluid flow lumen 29, (c) distal channel-fluid-flow guide end opening 127B, and (d) main-fluid-flow guide lateral opening 30 to outside main fluid flow lumen 29.

This insertion of secondary guidewire 50B is typically performed while main stent-graft 22 is in the radially-compressed delivery state and while main stent-graft 22 (and main delivery catheter 80A) is outside the patient's body, such as shown in FIG. 6B. Typically, but not necessarily, second end portion 58B of secondary guidewire 50B (which passes out of vasculature 54 at third vascular access site 52C) is inserted into the secondary-guidewire-tube lumen of secondary guidewire tube 140.

Typically, secondary guidewire 50B is advanced along (e.g., through) the delivery system to a delivery handle of the delivery system.

In addition, also as shown in FIG. 6B, typically primary guidewire 50A is inserted into guidewire longitudinal lumen 79 of main inner shaft 78 of the delivery system, also typically while main stent-graft 22 (and main delivery catheter 80A) is outside the patient's body. Typically, but not necessarily, second end portion 58A of primary guidewire 50A (which passes out of vasculature 54 at third vascular access site 52C) is inserted into guidewire longitudinal lumen 79.

In some applications, such as shown in FIG. 6C, secondary guidewire tube 140 is removed from main stent-graft 22 (and main delivery catheter 80A, as well as entirely from the delivery system). As a result of this removal, secondary guidewire 50B remains positioned passing sequentially through (a) proximal main-fluid-flow guide end opening 27A, (b) longitudinal portion 142 of main fluid flow lumen 29, (c) distal channel-fluid-flow guide end opening 127B, (d) main-fluid-flow guide lateral opening 30 to outside main fluid flow lumen 29, and (e) and out of a proximal catheter end 83 of main delivery catheter 80A (optionally into a handle of the delivery system (not shown)).

For some applications, this removal of secondary guidewire tube 140 from main stent-graft 22 is performed while main stent-graft 22 (and main delivery catheter 80A) is outside the patient's body, such as shown in FIG. 6C. Alternatively, this removal is performed after the main stent-graft has been introduced into vasculature 54, such as shown in FIG. 2C.

The method continues as described hereinabove with reference to FIGS. 2C-O: main stent-graft 22 is introduced into vasculature 54 while (a) main stent-graft 22 is in the radially-compressed delivery state, and (b) internal support channel 120 is in the collapsed state, in which distal channel-fluid-flow guide end opening 127B faces radially inward within main fluid flow lumen 29. Thereafter, main stent-graft 22 is transitioned to the radially-expanded deployment state, as shown in the transition between FIG. 2C and FIG. 2D. Also as shown in FIG. 2D, internal support channel 120 is configured to automatically transition to the expanded state when not constrained in the collapsed state by main stent-graft 22 and not constrained in the collapsed state by secondary guidewire tube 140. As described above, when internal support channel is in the expanded state, distal channel-fluid-flow guide end opening 127B faces at least partially distally within main fluid flow guide 28. (As shown in FIG. 2D, secondary guidewire 50B does not constrain internal support channel 120, even though secondary guidewire 50B passes loosely through internal support channel 120.) When internal support channel 120 is in the expanded state, distal channel-fluid-flow guide end opening 127B faces at least partially distally within main fluid flow guide 28, also as shown in FIGS. 2D-O. As described hereinabove with reference to FIGS. 2M-N, branching stent-graft 24 is deployed by advancing branching stent-graft 24 over secondary guidewire 50B (a) from first vascular access site 52A to within main stent-graft 22 (via distal main-fluid-flow guide end opening 27B), (b) from within main stent-graft 22, and (c) partially into second branch 70 via main-fluid-flow guide lateral opening 30. As a result, branching stent-graft 24 is partially in second branch 70 external to main-fluid-flow guide lateral opening 30 and partially inside main stent-graft 22. In order to advance branching stent-graft 24 from within main stent-graft 22 and partially into second branch 70, branching stent-graft 24 is advanced (a) from within main stent-graft 22 into distal channel-fluid-flow guide end opening 127B of channel fluid flow guide 128 of internal support channel 120, (b) through channel fluid flow guide 128, and (c) out of main-fluid-flow guide lateral opening 30 via proximal channel-fluid-flow end opening 127A. The insertion of branching stent-graft 24 from within main stent-graft 22 into distal channel-fluid-flow guide end opening 127B of channel fluid flow guide 128 is facilitated by the at least partially distal facing of distal channel-fluid-flow guide end opening 127B within main fluid flow guide 28. Typically, branching stent-graft 24 is disposed in its radially-compressed delivery state within a portion of branching delivery catheter 80B during the above-described advancing of branching stent-graft 24.

Reference is now made to FIG. 7, which is a schematic illustration of a configuration of main stent-graft 22 disposed in the radially-compressed delivery state within a portion of main delivery catheter 80A, in accordance with an application of the present invention. (For clarity of illustration, main stent-graft 22 is shown slightly expanded in FIG. 7, even though it is in practice generally more radially-compressed when in this delivery state.)

In this configuration, secondary guidewire 50B is removably pre-positioned passing sequentially through (a) proximal main-fluid-flow guide end opening 27A, (b) longitudinal portion 142 of main fluid flow lumen 29, (c) distal channel-fluid-flow guide end opening 127B, (d) main-fluid-flow guide lateral opening 30 to outside main fluid flow lumen 29, and (e) and out of a proximal catheter end 83 of main delivery catheter 80A (optionally into a handle of the delivery system (not shown)).

In an embodiment, techniques and apparatus described in one or more of the following applications, which are incorporated herein by reference, are combined with techniques and apparatus described herein:
U.S. Pat. No. 8,317,856 to Benary et al.
U.S. Pat. No. 8,870,938 to Shalev et al.
U.S. Pat. No. 8,945,203 to Shalev et al.
U.S. Pat. No. 9,993,360 to Shalev et al.
U.S. Pat. No. 9,668,892 to Shalev
US Patent Application Publication 2016/0193029 to Shalev
U.S. Pat. No. 10,603,197 to Marmur et al.
U.S. Pat. No. 10,485,684 to Marmur et al.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A stent-graft system comprising:
(i) a main stent-graft, which is configured to assume a radially-compressed delivery state and a radially-expanded deployment state, and which comprises:
a flexible main-stent-graft stent member and a generally tubular main fluid flow guide, which (a) is securely attached to and covers at least a portion of the main-stent-graft stent member, and (b) is shaped so as to define proximal and distal main-fluid-flow guide end openings, a main fluid flow lumen therebetween, and a main-fluid-flow guide lateral opening; and
an internal support channel, which (a) is configured to assume expanded and collapsed states, (b) is disposed within the main fluid flow guide, (c) comprises a generally tubular channel fluid flow guide, which is shaped so as to define (1) proximal and distal channel-fluid-flow end openings and (2) when the internal support channel is in the expanded state, a channel lumen between the proximal and the distal channel-fluid-flow end openings, wherein:
(A) when the internal support channel is in the collapsed state, the distal channel-fluid-flow guide end opening faces radially inward, and
(B) when the internal support channel is in the expanded state and the main stent-graft is in the radially-expanded deployment state, the distal channel-fluid-flow guide end opening faces at least partially distally within the main fluid flow guide,
wherein the proximal channel-fluid-flow end opening is sealingly coupled to a perimeter of the main-fluid-flow guide lateral opening, such that the channel lumen is in fluid communication with outside the main fluid flow guide via the main-fluid-flow guide lateral opening when the internal support channel is in the expanded state; and
(ii) an elongate member, which is removably positioned passing sequentially through (a) the proximal main-fluid-flow guide end opening, (b) a longitudinal portion of main fluid flow lumen, (c) the distal channel-fluid-flow guide end opening, and (d) the main-fluid-flow guide lateral opening to outside the main fluid flow lumen,
wherein the internal support channel is configured to automatically transition to expanded state when not constrained in the collapsed state by the main stent-graft and constrained in the collapsed state by the elongate member.

2. The stent-graft system according to claim 1, for use with a secondary guidewire, wherein the elongate member comprises a secondary guidewire tube, which is shaped so as to define a secondary-guidewire-tube lumen for insertion therethrough of the secondary guidewire.

3. The stent-graft system according to claim 2,
wherein a longitudinal portion of the secondary guidewire tube includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire tube is neither folded nor kinked.

4. The stent-graft system according to claim 2,
wherein a longitudinal portion of the secondary guidewire tube includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion defines a directional path from the proximal main-fluid-flow guide end opening to the distal end of the second longitudinal sub-portion, and the directional path does not include any proximally-directed portions.

5. The stent-graft system according to claim 2,
wherein a longitudinal portion of the secondary guidewire tube includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire tube has a smallest radius of curvature, at any location along the longitudinal portion, that is at least 0.15 cm.

6. The stent-graft system according to claim 5, wherein the smallest radius of curvature is at least 1 cm.

7. The stent-graft system according to claim 2, wherein the main stent-graft is configured such that the secondary guidewire tube is removable from the internal support channel while the main stent-graft is in the radially-compressed delivery state.

8. The stent-graft system according to claim 7, wherein the main stent-graft is configured such that the secondary guidewire tube is removable from the internal support channel while the main stent-graft is in the radially-compressed delivery state and the secondary guidewire is inserted through the secondary-guidewire-tube lumen.

9. The stent-graft system according to claim 1, wherein when the internal support channel is in the expanded state and the main stent-graft is in the radially-expanded deployment state, the distal channel-fluid-flow guide end opening is disposed distal to the main-fluid-flow guide lateral opening.

10. The stent-graft system according to claim 1, wherein the internal support channel is configured such that when the internal support channel is in the collapsed state, a proximal-most point of the distal channel-fluid-flow guide end opening is disposed proximal of a distal-most point of the main-fluid-flow guide lateral opening.

11. The stent-graft system according to claim 1, wherein the internal support channel is configured such that, during the automatic transition of the internal support channel from the collapsed state to the expanded state, a best-fit plane defined by the distal channel-fluid-flow guide end opening automatically swings:
from the distal channel-fluid-flow guide end opening facing at least partially radially inward,
to the distal channel-fluid-flow guide end opening facing at least partially distally.

12. The stent-graft system according to claim 1,
wherein the main-stent-graft stent member comprises struts, and
wherein an external surface of the internal support channel is coupled to an internal surface of the main fluid flow guide by being coupled to one or more of the struts of the main-stent-graft stent member, such that the internal support channel runs alongside the internal surface of the main fluid flow guide distally from the main-fluid-flow guide lateral opening.

13. The stent-graft system according to claim 1, wherein the elongate member comprises a secondary guidewire.

14. The stent-graft system according to claim 13,
wherein a longitudinal portion of the secondary guidewire includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire is neither folded nor kinked.

15. The stent-graft system according to claim 13,
wherein a longitudinal portion of the secondary guidewire includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion defines a directional path from the proximal main-fluid-flow guide end opening to the distal end of the second longitudinal sub-portion, and the directional path does not include any proximally-directed portions.

16. The stent-graft system according to claim 13,
wherein a longitudinal portion of the secondary guidewire includes (a) a first longitudinal sub-portion extending between the proximal main-fluid-flow guide end opening and the main-fluid-flow guide lateral opening, via the distal channel-fluid-flow guide end opening, and (b) a second longitudinal sub-portion extending out of the main-fluid-flow guide lateral opening, outside the main fluid flow lumen, and having a length of 1 cm, measured from the main-fluid-flow guide lateral opening to a distal end of the second longitudinal sub-portion, and
wherein when the distal end of the second longitudinal sub-portion is held in contact with an external surface of the main stent-graft while the main stent-graft is in the radially-compressed delivery state, the longitudinal portion of the secondary guidewire has a smallest radius of curvature, at any location along the longitudinal portion, that is at least 0.15 cm.

17. The stent-graft system according to claim 16, wherein the smallest radius of curvature is at least 1 cm.

18. The stent-graft system according to any one of claims 1-17, wherein the internal support channel further comprises a flexible channel stent member to which the channel fluid flow guide is securely attached.

19. The stent-graft system according to claim 18, wherein the channel stent member comprises one or more stent struts, which are shaped so as to define a tongue-shaped stent strut that is securely attached to a portion of a perimeter of the distal channel-fluid-flow guide end opening.

20. The stent-graft system according to claim 19, wherein the tongue-shaped stent strut is configured such that, during the automatic transition of the internal support channel from the collapsed state to the expanded state, a tip of the tongue-shaped stent strut automatically swings along a curved path from facing proximally to facing less proximally.

21. The stent-graft system according to claim 19, wherein a minimal surface defined by the tongue-shaped stent strut faces:
at least partially distally when the internal support channel is in the expanded state, and
at least partially radially inward when the internal support channel is in the collapsed state.

22. The stent-graft system according to claim 21, wherein the tongue-shaped stent strut is configured such that, during the automatic transition of the internal support channel to the expanded state, the tongue-shaped stent strut automatically swings:
from the minimal surface facing at least partially radially inward,
to the minimal surface facing at least partially distally.

23. The stent-graft system according to any one of claims 1-17,
wherein the stent-graft system further comprises a main delivery catheter having proximal and distal catheter ends,
wherein the main stent-graft is removably disposed within the main delivery catheter such that the main delivery catheter constrains the main stent-graft in the radially-compressed delivery state with the distal main-fluid-flow guide end opening facing the distal catheter end, thereby constraining the internal support channel in the collapsed state, and
wherein the elongate member is removably positioned passing sequentially through (a) the proximal main-fluid-flow guide end opening, (b) the longitudinal portion of the main fluid flow lumen, (c) the distal channel-fluid-flow guide end opening, (d) the main-fluid-flow guide lateral opening to outside the main fluid flow lumen within the main delivery catheter, and (e) the distal catheter end.

24. The stent-graft system according to claim 23, wherein a longitudinal portion of the elongate member extends between the proximal main-fluid-flow guide end opening of the radially-compressed main stent-graft and the distal catheter end, and has a smallest radius of curvature, at any location along the longitudinal portion, that is at least 0.15 cm.

25. The stent-graft system according to claim 24, wherein the smallest radius of curvature is at least 1 cm.

26. The stent-graft system according to claim 23, wherein a longitudinal portion of the elongate member extends between the proximal main-fluid-flow guide end opening of the radially-compressed main stent-graft and the distal catheter end, and is neither folded nor kinked.

27. The stent-graft system according to claim 23, wherein a longitudinal portion of the elongate member defines a directional path from the proximal main-fluid-flow guide end opening of the radially-compressed main stent-graft to the distal catheter end, and the directional path does not include any proximally-directed portions.

28. A method comprising:
providing a main stent-graft, which is configured to assume a radially-compressed delivery state and a radially-expanded deployment state, and which includes:
a flexible main-stent-graft stent member and a generally tubular main fluid flow guide, which (a) is securely attached to and covers at least a portion of the main-stent-graft stent member, and (b) is shaped so as to define proximal and distal main-fluid-flow guide end openings, a main fluid flow lumen therebetween, and a main-fluid-flow guide lateral opening; and
an internal support channel, which (a) is configured to assume expanded and collapsed states, (b) is disposed within the main fluid flow guide, (c) includes a generally tubular channel fluid flow guide, which is shaped so as to define (1) proximal and distal channel-fluid-flow guide end openings and (2) when the internal support channel is in the expanded state, a channel lumen between the proximal and the distal channel-fluid-flow guide end openings,
wherein the proximal channel-fluid-flow guide end opening is sealingly coupled to a perimeter of the main-fluid-flow guide lateral opening, such that the channel lumen is in fluid communication with outside the main fluid flow guide via the main-fluid-flow guide lateral opening when the internal support channel is in the expanded state;
providing an elongate member that is removably positioned passing sequentially through (a) the proximal main-fluid-flow guide end opening, (b) a longitudinal portion of the main fluid flow lumen, (c) the distal channel-fluid-flow guide end opening, and (d) the main-fluid-flow guide lateral opening to outside the main fluid flow lumen;
introducing the main stent-graft into vasculature of a patient while (a) the main stent-graft is in the radially-compressed delivery state, and (b) the internal support channel is in the collapsed state, in which the distal channel-fluid-flow guide end opening faces radially inward; and
thereafter, transitioning the main stent-graft to the radially-expanded deployment state,
wherein the internal support channel is configured to automatically transition to the expanded state when not constrained in the collapsed state by the main stent-graft and not constrained in the collapsed state by the elongate member, and wherein when the internal support channel is in the expanded state, the distal channel-fluid-flow guide end opening faces at least partially distally within the main fluid flow guide.

29. A method comprising:
endovascularly introducing a vascular implant into vasculature of a patient through vascular access site that is downstream of a first branch of an aortic arch;
thereafter, advancing the vascular implant through the first branch to the aortic arch;
thereafter, advancing the vascular implant from the aortic arch at least partially into a second branch of the aortic arch; and
thereafter, deploying the vascular implant at least partially in the second branch.

30. A method comprising:
deploying a guidewire between (i) a first vascular access site that is downstream of first branch of an aortic arch of a patient and (ii) a second vascular access site that is downstream of a second branch of the aortic arch, such that the guidewire is positioned partially within the first branch, partially within the aortic arch, and partially within the second branch;
advancing a vascular implant over the guidewire; and
deploying the vascular implant within vasculature of the patient.
